(19) United States
(12) Patent Application Publication
(10) Pub. No.: US 2014/0336676 A1
(43) Pub. Date: Nov. 13, 2014

(54) TISSUE LIGATION DEVICES AND METHODS THEREFOR

(71) Applicant: SENTREHEART, INC., Redwood City, CA (US)

(72) Inventors: Russell PONG, Newark, CA (US); Robert L. CLARK, III, Hayward, CA (US); Gregory W. FUNG, Redwood Shores, CA (US); Arnold M. ESCANO, Santa Clara, CA (US)

(73) Assignee: SENTREHEART, INC., Redwood City, CA (US)

(21) Appl. No.: 14/272,478

(22) Filed: May 7, 2014

Related U.S. Application Data
(60) Provisional application No. 61/821,153, filed on May 8, 2013.

(51) Int. Cl. A61B 17/12

(52) U.S. Cl. CPC A61B 17/12013
USPC 606/144

(57) ABSTRACT

Described here are closure devices and methods for ligating tissue, such as the left atrial appendage. The closure devices may comprise a snare loop assembly comprising a snare and a suture loop releasably attached thereto, and a snare control. The closure devices may further comprise one or more reference markings, wherein the relative positioning between a portion of the snare control and the reference markings provides an indication of the diameter of the snare loop assembly. In some variations, when the snare loop assembly is closed around a tissue such as the left atrial appendage, the measured diameter of the snare loop assembly may help a user determine whether the snare loop assembly has been properly positioned relative to the tissue, as well as the approximate size that a suture loop may have when released from the snare loop assembly.

### CROSS-REFERENCE TO RELATED APPLICATIONS

This application claims priority to U.S. Provisional Patent Application Ser. No. 61/821,153, filed on May 8, 2013, which is incorporated by reference herein in its entirety.

### FIELD

This invention relates generally to devices and methods for ligating tissue, such as the left atrial appendage.

### BACKGROUND

Atrial fibrillation is a common problem that afflicts millions of patients. Atrial fibrillation often results in the formation of a thrombus, or clot, in the appendage of the left atrium. This presents a problem, inasmuch as the thrombus can dislodge and embolize to distant organs, which may result in adverse events such as a stroke. For this reason, most patients with atrial fibrillation are treated with one or more blood thinners to help prevent the formation of a thrombus. Blood thinners, however, can present health risks of their own, especially in the elderly. These risks, such as bleeding, often require a user to make significant lifestyle changes.

Several methods have been developed to address the potential problem of thrombus formation in the left atrial appendage. One such method includes suturing the left atrial appendage along the base or ostial neck where it joins the atrial chamber. In this way, blood flow into the atrial appendage is cut off, eliminating the risk of thrombus formation therein. This is typically done through open-heart surgery, which limits the availability of the procedure to those who are at a particularly high risk, or who are otherwise undergoing an open-heart procedure. In addition, open-heart surgery requires general anesthesia and has a number of well-known risks, making it less desirable.

Other methods have also been investigated. These methods include methods of stapling the base of the appendage and methods of filling the appendage with a space occupying or occluding member. Stapling is not preferred given the fragility of the appendage and its tendency to rupture, while occlusion devices may not effectively prevent all blood flow into the appendage.

Additional devices and methods for closing the left atrial appendage or other suitable tissues would therefore be desirable. In particular, devices and methods for closing the left atrial appendage using minimally invasive, intravascular, or a combination of these techniques, would be desirable in order to avoid the need for opening the chest. Of course, additional devices for use in open surgical procedures are desirable as well, especially when those devices offer additional advantages over standard devices.

### BRIEF SUMMARY

Described here are closure devices and methods for ligating tissue. Generally, the closure devices include a snare loop assembly which may be selectively closed around tissue, and may be configured to deliver a suture loop therefrom to hold the tissue in a closed configuration. The snare loop assembly generally comprises a snare and a suture loop, and a portion of the snare loop assembly may extend from an elongate body to define a loop that may be positioned around tissue. The snare may be moveable relative to the elongate body to increase or decrease the diameter of the loop defined by the snare loop assembly, which may allow the snare loop assembly to be closed around tissue.

In some variations, the closure device may comprise a handle having a snare control. The snare control may be configured to move the snare relative to the elongate body to alter the diameter of the loop defined by the snare loop assembly. The handle may further comprise one or more reference markings, wherein the relative positioning between a portion of the snare control and the reference markings provides an indication of the diameter of the snare loop assembly. When the snare loop assembly is closed around a tissue such as the left atrial appendage, the measured diameter of the snare loop assembly may help a user determine whether the snare loop assembly has been properly positioned relative to the tissue, as well as the approximate size that a suture loop may have when released from the snare loop assembly.

In some variations, the measured diameter of the snare loop assembly may assist a user in further closing the snare loop assembly and/or tightening a suture loop. For example, in some variations, the snare control may comprise a control knob which may be configured to apply tension to the snare to reduce the diameter of the snare loop assembly. The control knob may comprise one or more reference markings which may be indicative of the force applied by the control knob to the snare. In these variations, a user may select the force to be applied by the control knob based on the diameter of the snare loop assembly as it closes tissue. In other variations, a tensioning device may be used to tighten the suture loop, and the tensioning device may comprise one or more reference markings which may be indicative of the force applied by the tensioning device to the suture loop. In these variations, a user may select the force to be applied by the tensioning device based on the diameter of the snare loop assembly as it closes tissue.

### BRIEF DESCRIPTION OF THE DRAWINGS

FIG. 1 depicts a distal end of an illustrative variation of a closure device having a snare loop assembly.

FIG. 2 shows a cross-sectional side view of the closure device of FIG. 1.

FIG. 3 is a perspective view of an illustrative closure device as described here.

FIGS. 4A and 4B depict perspective views of a proximal portion of an illustrative closure device as described here. FIG. 4C shows a top view of a portion of a variation of a handle suitable for use with the closure devices described here.

FIG. 5A depicts a cross-sectional bottom view of a variation of a handle suitable for use with the closure devices described here. FIGS. 5B and 5C show cross-sectional perspective views of the handle of FIG. 5A.

FIGS. 6A and 6B show a perspective view and a bottom cross-sectional view, respectively, of a variation of a handle suitable for use with the closure devices described here.

FIGS. 7A and 7B show a perspective view and a cross-sectional perspective view, respectively, of an illustrative variation of a handle suitable for use with the closure devices described here.

FIG. 8 shows a variation of the closure devices described here and a tensioning device for tensioning a suture loop of the closure device.

FIGS. 9A and 9B depict perspective views of a variation of a handle suitable for use with the closure devices described here. FIG. 9C shows a variation of a reference marker suitable for use with the handle of FIGS. 9A and 9B. FIGS. 9D and 9E show a bottom perspective view and an exploded bottom perspective view, respectively, of the handle of FIGS. 9A and 9B.

FIGS. 10A-10D depict cross-sectional side views of a distal portion of variations of the closure devices described here.

FIGS. 11A-11C depict cross-sectional side views of a distal portion of a variation of the closure devices described here.

FIGS. 12 and 13 depict distal portions of variations of the closure devices described here.

FIGS. 14A and 14B depict cross-sectional side views of a distal portion of variations of the closure devices described here.

FIGS. 15-18 depict schematic representations of methods of using the closure devices described here.

### DETAILED DESCRIPTION

Described here are closure devices and methods for closing tissues using one or more closure devices. Generally, the closure devices comprise an elongate body and a snare loop assembly, such as those described in more detail with respect to U.S. patent application Ser. No. 13/490,919, entitled “Tissue Ligation Devices and Tensioning Devices Therefor” and filed on Jun. 7, 2012, the content of which is incorporated by reference herein in its entirety. The snare loop assembly is generally configured to be closed around tissue to temporarily or permanently close, ligate, or otherwise tighten tissue, and may be further configured to deliver a suture loop therefrom to hold or otherwise maintain the tissue in the closed configuration. The suture loop may have an adjustable diameter, and the closure devices are generally configured to help control the size of the diameter of the suture loop that is released from the snare loop assembly, as will be discussed in more detail below. While the devices and methods described here are discussed in relation to ligating the left atrial appendage, it should be appreciated that the devices and methods described here may be used to close any suitable tissue.

### I. Devices

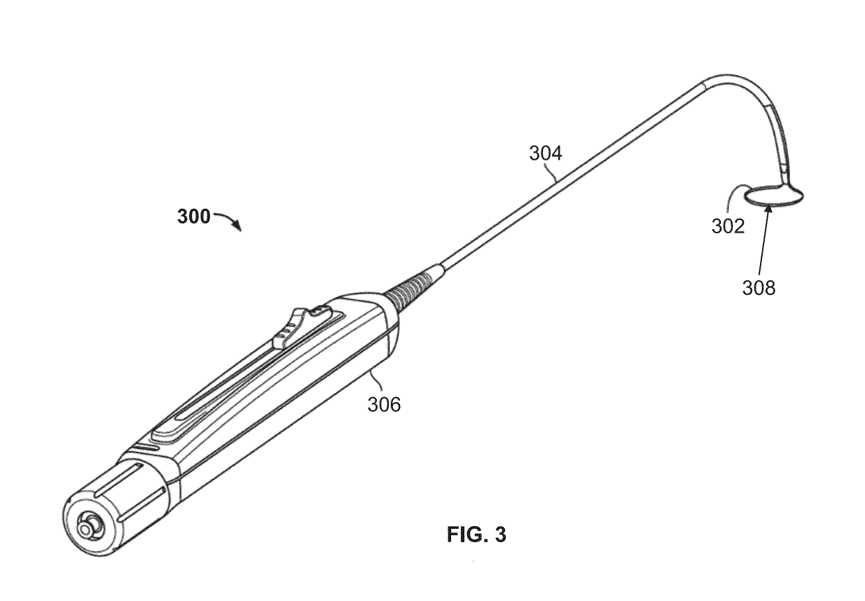

FIG. 3 depicts one illustrative variation of a closure device (300) as described here. As shown there, the closure device (300) may comprise a snare loop assembly (302), an elongate body (304), and a handle (306). Generally, a portion of the snare loop assembly (302) extends from a distal portion of the elongate body (304) to form a continuous loop (308), which may allow the snare loop assembly (302) and the elongate body (304) to encircle tissue placed in the loop (308). The handle (306) may be used to control and actuate the snare loop assembly (302) through the elongate body (304) in order to increase or decrease the size of the loop (308). For example, the handle (306) may advance a portion of the snare loop assembly (302) out of the elongate body (304) to increase the size of the loop (308), or may withdraw a portion of the snare loop assembly (302) into the elongate body (304) to decrease the size of the loop (308). Accordingly, the size of the loop (308) may be increased to allow the snare loop assembly (302) to be placed around tissue. Once around tissue, the size of the loop (308) may be decreased to ligate tissue.

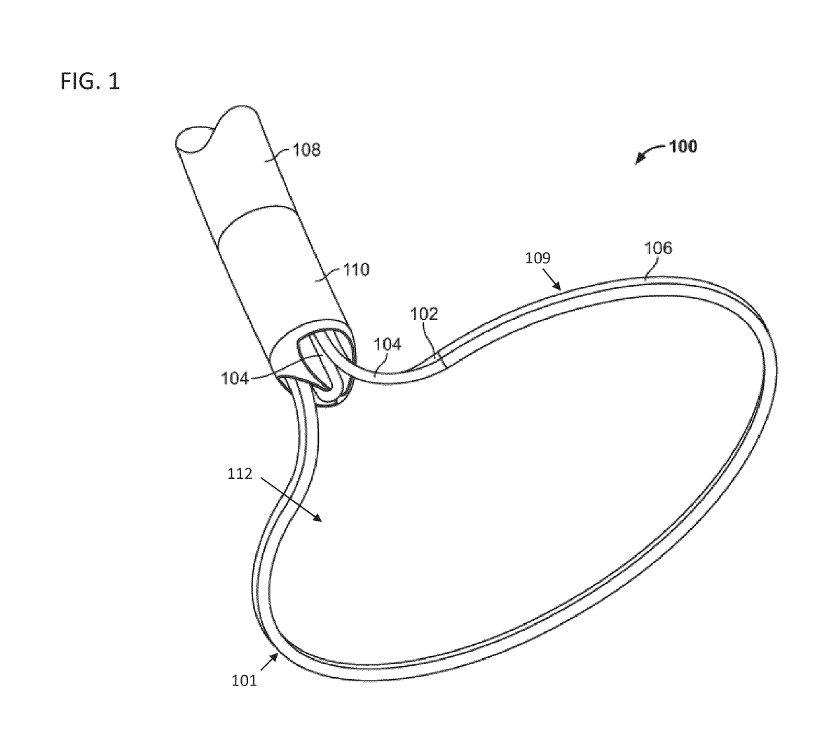

The snare loop assemblies of the closure devices described here generally comprise a snare and a suture loop releasably coupled thereto. For example, FIG. 1 shows a distal portion of an illustrative variation of a closure device (100) comprising a snare loop assembly (101) and an elongate body (108) having a tip (110). As shown there, the snare loop assembly (101) may comprise a snare (102), a suture loop (104), and a retention member (106), and may be disposed relative to the elongate body (108) such that at least a portion of the snare loop assembly (101) extends from the elongate body (108) (e.g., out of tip (110)). The snare loop assembly (101) is shown in FIG. 1 in an open configuration, and the portion of snare loop assembly (101) extending out of elongate body (104) may form a loop (109) having an aperture (112) therethrough, such as discussed above. The loop (109) and corresponding aperture (112) may be defined by one or more components of the snare loop assembly (101) (e.g., the snare), and may be suitable for encircling tissue such as the left atrial appendage.

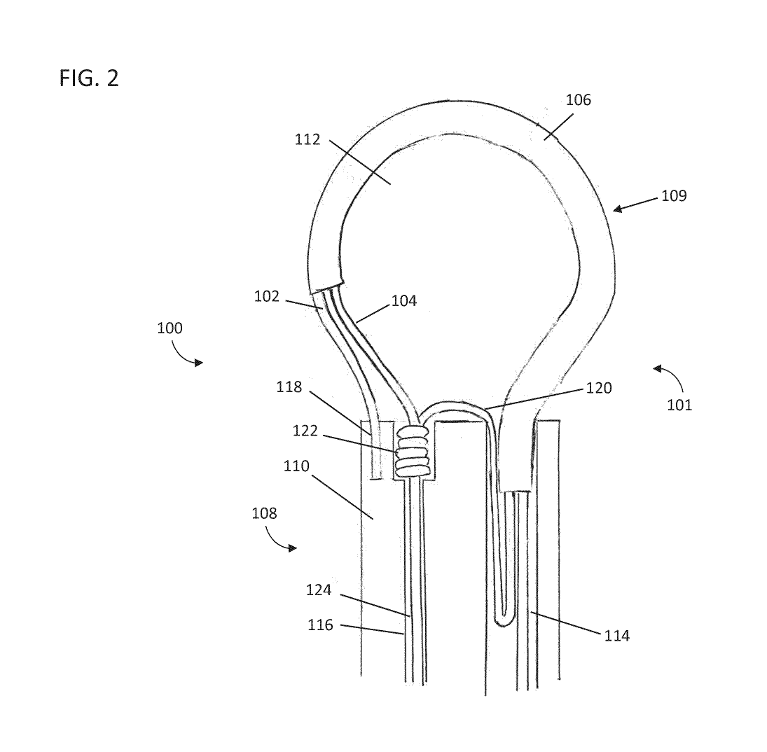

Generally, the snare (102) may be actuated (e.g., by a portion of a handle or other control portion of the closure device) to control the size of the loop (109) of the snare loop assembly (101). For example, FIG. 2 shows a cross-sectional side view of the closure device (100). As shown there, the elongate body (108) may comprise a first lumen (114) and a second lumen (116). One end (118) of the snare (102) may be fixedly attached to the elongate body (108) (e.g., attached to the tip (110)), while a second end (not shown) of the snare may pass through the first lumen (114), where it may be operatively attached to a snare control (not shown). The snare control may be configured to advance or retract the snare (102) relative to the elongate body (108), as will be discussed in more detail below, which may control the amount of the snare (102) (and with it, the snare loop assembly (101)) extending from the elongate body (108). This in turn may control the size of the loop (109) of the snare loop assembly (101).

As mentioned above, a suture loop (104) may be releasably connected to the snare (102). For example, as shown in FIGS. 1 and 2, the suture loop (104) may be releasably coupled to the snare (102) via a retention member (106). The retention member (106) may be any suitable structure, such as a dual-lumen tube or one or more of the retention members described in U.S. patent application Ser. No. 13/490,919, which was previously incorporated by reference in its entirety. The suture loop (104) may be initially configured to have a diameter larger than that of the snare loop assembly (101) when the snare loop assembly (101) is opened (excess suture of the suture loop (104) may be housed in the elongate body (108), such as described in U.S. patent application Ser. No. 13/490,919, which was previously incorporated by reference in its entirety). The suture loop (104) may be tightened to reduce the diameter of the suture loop (104). When the diameter of the suture loop (104) is reduced past the diameter of the loop (109) of the snare loop assembly (101), the suture loop (104) may disengage and be released from the snare loop assembly (101). For example, tightening the suture loop (104) may cause the suture loop (104) to pull or tear through one or more walls, slits, prongs, arms or the like of the retention member (106) to break the connection between the suture loop (104) and the retention member (106).

Generally, the suture loop (104) may comprise a loop portion (120), a suture knot (122) and a tail (124). As shown in FIG. 2, the suture knot (122) may be temporarily held at least partially within the tip (110) of the elongate body (108). Suture of the loop portion (120) may be pulled through suture knot (122) to reduce the diameter of the loop portion (120). The suture tail may extend through the elongate body (108) (e.g., through the second lumen (116) of the elongate body (108)), and may be operatively attached to a suture control (not shown). The suture control may be used to pull the suture tail (124), which in turn may reduce the diameter of the loop portion (120) of the suture loop. When the snare (102) is advanced or withdrawn relative to the first lumen (114) of the elongate body (108), a portion of the suture loop (104) and the retention member (106) may also be advanced out of or withdrawn into the first lumen (114) of the elongate body (108). The suture knot (120) is preferably a one-way knot (e.g., a slip-knot), which allows the suture loop to maintain its diameter as the suture loop (104) is tightened. Additionally or alternatively, the suture loop (104) may comprise one or more unidirectional locking structures (such as those described in U.S. patent application Ser. No. 13/490,919, which was previously incorporated by reference in its entirety) which may help prevent the loop portion (120) from increasing in diameter (e.g., in response to expansive forces provided by the ligated tissue) of the suture loop (104) after it is tightened. Generally, referring to FIGS. 1 and 2, the moveable end of the snare (102) may be advanced relative to the elongate body (108) to increase the diameter of the loop (109) of the snare loop assembly (101) to “open” the snare loop assembly. In some variations, the closure device (100) may be advanced to the left atrial appendage with the loop (109) in the “open” configuration such that it is unnecessary to increase the diameter of the loop (109) at the left atrial appendage. With the snare loop assembly in an open configuration, the loop (109) may be placed around the target tissue to encircle the tissue. The moveable end of the snare (102) may be withdrawn relative to the elongate body (108) to decrease the diameter of the loop (109), which may close the snare loop assembly (101) around the tissue. With the tissue held in a closed configuration by the snare (102) and the snare loop assembly (101), the suture loop (104) may be tightened (i.e., the diameter of the loop portion (120) may be reduced by pulling the tail (124) relative to the suture knot (122)) to release the suture loop (104) from the snare loop assembly (101). Once released, the suture loop (104) may hold the tissue in a ligated configuration, and the remaining portions of the closure device (100) may be removed. In some instances, the suture loop may be further tightened to reduce the diameter of the suture loop, as will be discussed in more detail below.

Generally, when a closure device is used to ligate the left atrial appendage as described immediately above, closure of the left atrial appendage may help prevent blood from passing between the left atrium and the left atrial appendage through the ostium of the left atrial appendage. Generally, placement of the suture loop around the left atrial appendage may maintain closure of the left atrial appendage over time. Occasionally, however, repositioning or thinning of the tissue of the left atrial appendage may result in a slight opening of the ostium of the left atrial appendage, which may cause blood to leak between the left atrial appendage and the left atrium. Larger leaks (e.g., openings in the ostium of the left atrial appendage of about 5 mm or greater) may cause concern that clots that form or may have formed inside of the left atrial appendage may possibly enter the atrium through the leak. Accordingly, the closure devices and methods described here may be configured to close tissue such as the left atrial appendage in a manner that reduces the likelihood of leak formation and/or reduces the size of possible leaks that may form.

For example, in some instances it may be desirable to ensure that the suture loop delivered by the snare loop assembly is below a certain diameter when delivered. Because the suture loop should not increase in diameter after delivery from the snare loop assembly, the diameter of the suture loop may set a maximum possible size for any leaks that may form. For example, if a suture loop is tightened to 12 mm around the left atrial appendage, the size of any leak that may occur through the suture loop would not exceed 12 mm as long as the suture loop remains in place (although any leak should be smaller than 12 mm, as the tissue of the left atrial appendage captured by the suture loop will also limit the possible size of a leak). Similarly, if the suture loop is tightened to 4 mm around the left atrial appendage, the size of any leak that may occur through the suture loop would not exceed 4 mm. Accordingly, reducing the diameter of the suture loop reduces the possible size of any leaks that may develop. For example, it may be desirable to place a suture loop having a diameter less than or equal to 6 mm, 5 mm, 4 mm or 3 mm.

In instances where the suture loop is a one-way suture loop (as discussed above), it may not be possible to re-open the suture loop. It may be difficult for a user to know whether the suture loop will be able to achieve a desired closure diameter upon tightening of the suture loop, and the one-way nature of the suture loop may prevent a user from tightening the suture loop at multiple locations. In the closure devices described here, the closure device may be configured to provide a measurement of the amount of closure of the snare loop assembly, which may provide an indication of how much tissue is captured by the snare loop assembly and may also inform the user of whether a suture loop may be able to be tightened to a certain diameter before actually tightening the suture loop.

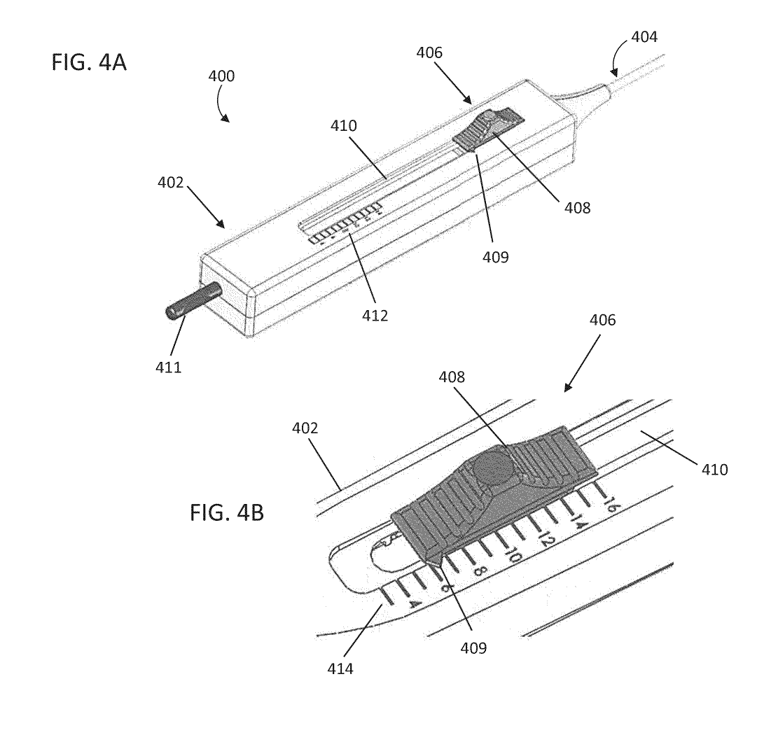

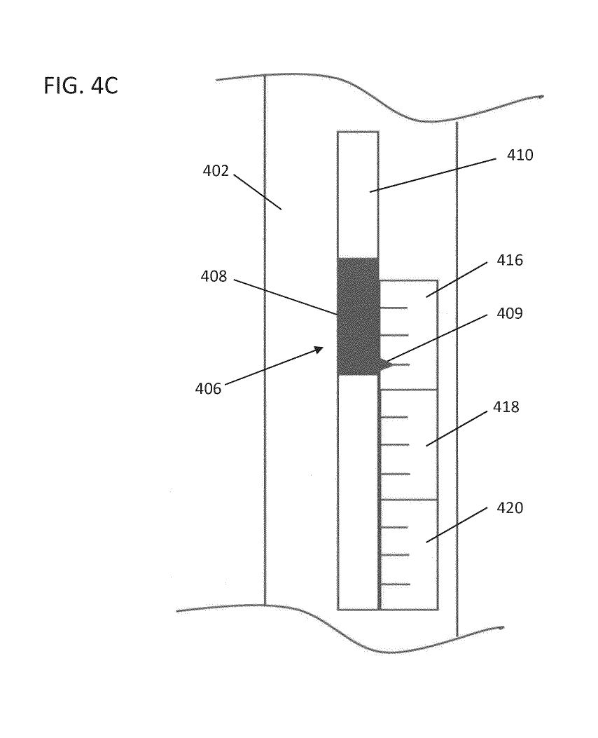

For example, FIGS. 4A and 4B show a variation of a perspective view of a proximal portion of a closure device (400), which may be used with a snare loop assembly (not shown) having a snare and a suture loop releasably attached to the snare, such as discussed above. Specifically, the closure device (400) may comprise a handle (402) and an elongate body (404), and the handle may comprise a snare control (406) and a suture control (411). The snare control (406) may be operatively attached to the snare, and may be configured to selectively advance and retract the snare relative to the elongate body (404), which may in turn open and close, respectively, the snare loop assembly. Similarly, the suture control (411) may be operatively attached to a tail portion of the suture loop, such that withdrawal of the suture control (411) may reduce the diameter of the loop portion of the suture loop. The handle (402) may further comprise a track (410) and one or more reference markings (412), which may be used in combination with the suture control (411) to provide a measurement of the diameter of the snare loop assembly.

For example, in the variation shown in FIGS. 4A and 4B, the snare control (406) may comprise a thumb slider (408) which is moveable along the track (410). The thumb slider (408) may comprise a pointer (409), and the position of the pointer (409) relative to the reference markings (412) may indicate the size of the loop of the snare loop assembly. In the variation shown in FIG. 4B, the reference markings (412) may comprise a plurality of tick marks (414), where each tick mark corresponds to a specific loop diameter of the snare loop assembly. Accordingly, when the snare loop assembly is positioned around a target tissue (such as the left atrial appendage), a user may withdraw the thumb slider (408) to close the snare loop assembly around the ensnared tissue. The positioning of the pointer (409) relative to the tick marks (414) may provide a measurement of the size of the snare loop assembly. The diameter of the snare loop assembly may also provide an indication of the amount of tissue captured by the snare loop assembly.

The diameter of the snare loop assembly may also provide an indication of the size of the suture loop that may be released from the snare loop assembly. Since the suture loop is generally tightened down to the diameter of the snare loop assembly to release the suture loop therefrom, the suture loop should have a diameter that is at least as small as the snare loop assembly. For example, if the snare loop assembly is closed around tissue to a diameter of 6 mm, the suture loop may be tightened to 6 mm or less to release the suture loop from the snare loop assembly (although it should be appreciated that the diameter of the suture loop may be further reduced after it is released from the snare loop assembly). Accordingly, by knowing the diameter of the snare loop assembly, a user may know the approximate size of the suture loop when it is released from the snare loop assembly.

The snare loop assembly diameter measurement may help a user control the size of the suture loop deployed therefrom. For example, when the closure devices described here are used to close the left atrial appendage, it may be desirable to close the left atrial appendage to a predetermined diameter with the snare loop assembly prior to releasing a suture loop therefrom. For example, in the variation of the snare control (406) shown in FIGS. 4A and 4B, the thumb slider (408) may be withdrawn until the pointer (409) reaches a reference marker (412) that corresponds to the desired snare loop assembly diameter. The diameter may be any suitable diameter, such as, for example, less than or equal to about 8 mm. This diameter may preferably be less than or equal to about 7 mm, about 6 mm, about 5 mm, about 4 mm, or about 3 mm. Once the snare loop assembly is closed to the predetermined diameter, the user may tighten and release the suture loop at a diameter less than or equal to the predetermined diameter.

In some instances, the snare loop assembly diameter measurement may allow a user to determine whether the snare loop assembly has been properly positioned relative to target tissue. When the closure devices described here are used to close the left atrial appendage, it may be desirable to place the snare loop assembly around the neck of the left atrial appendage, such that the left atrial appendage is closed near the ostium of the left atrial appendage. If the snare loop assembly is advanced too far, the snare loop assembly may capture atrial tissue, which may interfere with the ability of the snare loop assembly to be closed to a certain diameter. This may result in deployment of a larger suture loop, which in turn may increase the likelihood of a leak forming and/or may increase the size of a leak that forms. Conversely, if the snare loop assembly is not advanced far enough relative to the left atrial appendage, the snare loop assembly may only close off a portion of the left atrial appendage, which may still leave a portion of the left atrial appendage capable of receiving blood from the atrium. Accordingly, the amount of tissue captured by the snare loop assembly may provide an indication of the placement of the snare loop assembly relative to the ostium of the left atrial appendage.

For example, in the variation of the snare control (406) shown in FIGS. 4A and 4B, the thumb slider (408) may be withdrawn to close the snare loop assembly around the left atrial appendage. In some variations, a user may withdraw the thumb slider (408) until the tissue captured by the snare loop assembly significantly resists further withdrawal of the thumb slider (408). In other instances, the user may withdraw the thumb slider (408) until a predetermined force is applied to the snare (which may be measured by a force gauge in the snare control). When the snare loop assembly has been closed around the tissue, a user may use the relative positioning between the pointer (409) and the reference markings (412) to determine the diameter of the snare loop assembly (and with it, the amount of tissue captured by the snare loop assembly). If the diameter of the snare loop assembly is within a predetermined range (e.g., between about 7 mm and about 9 mm), the snare loop assembly may have captured an amount of tissue that is consistent with the typical anatomy of the neck of the left atrial appendage. This may provide an indication to a user that the snare loop assembly has been properly positioned relative to the ostium of the left atrial appendage, and a user may then deliver the suture loop to maintain closure of the left atrial appendage.

If the diameter of the snare loop assembly is above a certain level (e.g., greater than 9 mm), the snare loop assembly may have captured more tissue than is typical for the neck of the left atrial appendage. This may indicate that the snare loop assembly has ensnared atrial tissue in addition to tissue of the left atrial appendage (as discussed above), and in some instances the user may re-open and reposition the snare loop assembly. In other instances, the user may determine that the patient has atypical anatomy (e.g., a left atrial appendage having a larger-than-expected neck), and may choose to deploy the suture loop at this location. Conversely, if the diameter of the snare loop assembly is below a certain level (e.g., less than 7 mm), the snare loop assembly may have captured less tissue than is typical for the neck of the left atrial appendage. This may indicate that the snare loop assembly has been positioned too far away from the ostium of the left atrial appendage, and in some instances a user may re-open the snare loop assembly and reposition the snare loop assembly closer to the ostium of the left atrial appendage. In other instances, a user may determine that the patient has atypical left atrial appendage anatomy (e.g., a left atrial appendage having a smaller-than-expected neck), and may opt to deploy the suture loop at this location.

In some variations, a snare control may include one or more reference markings that are divided into one or more regions that correspond to one or more diameter ranges for the snare loop assembly. For example, FIG. 4C shows a top view of a variation of the handle (402) depicted in FIGS. 4A and 4B (identical components are labeled as such). As shown there, the reference markings (412) may be divided into a first region (416), a second region (418), and a third region (420). The regions may be visually distinct from each other. For example, in some variations, the regions may be color-coded such that each region is marked with a different color. Additionally or alternatively, each region may be boxed-off from the other regions. Additionally, while shown in FIG. 4C as having three regions, the reference markings (412) may be divided into any suitable number of regions.

Each region of the reference markings (412) may correspond to a different range of snare loop assembly diameters (e.g., the first region (416) may correspond to a first range of snare loop assembly diameters, the second region (418) may correspond to a second range of snare loop assembly diameters, and the third region (420) may correspond to a third range of snare loop assembly diameters). As shown in FIG. 4C, the first range of snare loop assembly diameters may include larger diameters than those of the second range, which in turn may be greater than those of the third range. In some variations, the ranges of the reference markings (412) may correspond to the values expected for typical left atrial appendage anatomy. For example, the first range of the first region (416) may correspond to values (e.g., greater than 9 mm, between 9 mm and 12 mm) that indicate that the snare loop assembly has captured more tissue than is expected at the neck of a typical left atrial appendage. The second range of the second region (418) may correspond to values (e.g., between 7 mm and 9 mm) that indicate that the snare loop assembly has captured tissue that is consistent with the neck of a typical left atrial appendage, and the first range of the first region (416) may correspond to values (e.g., less than 7 mm, between 2 mm and 7 mm) that indicate that the snare loop assembly has captured less tissue than is expected at the neck of a typical left atrial appendage. When closing the snare loop assembly, these ranges (in conjunction with the pointer (409) of the thumb slider (408)) may allow a user to determine whether to reposition the snare loop assembly and/or what size suture loop may be delivered from the snare loop assembly.

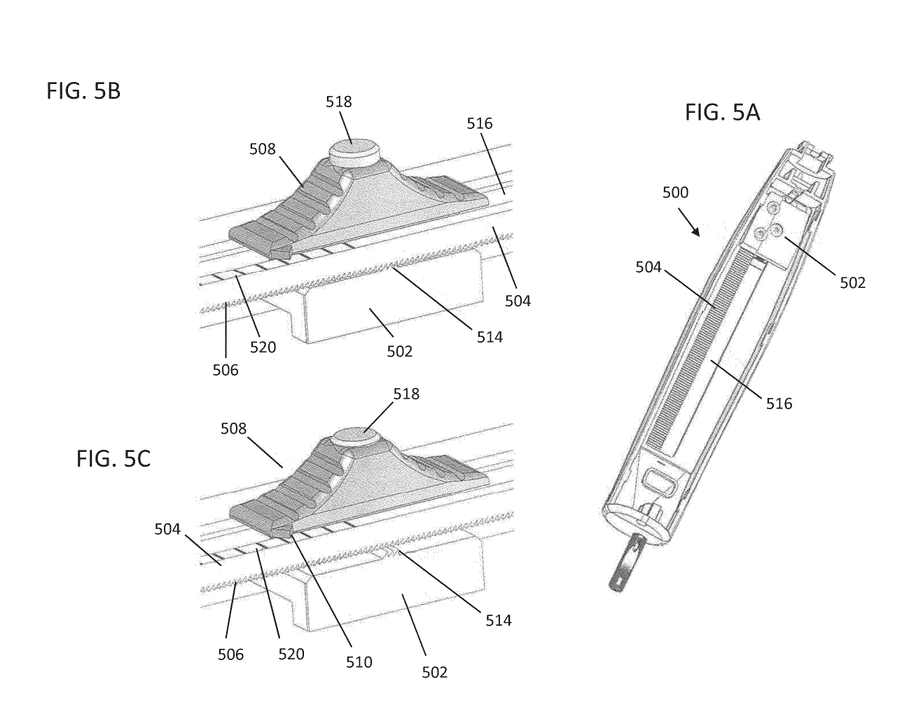

The snare control (406) may include any mechanism suitable to advance and/or withdraw the snare relative the elongate body to open and/or close the snare loop assembly. In variations where the snare control comprises a thumb slider (such as the thumb slider (408) shown in FIGS. 4A-4C), the thumb slider may be freely moved relative to the handle, or may be configured to be temporarily prevented from advancement and/or retraction. FIGS. 5A-5C depict one such variation of a handle (500) suitable for use with the closure devices described here. Specifically, FIG. 5A shows a cross-sectional bottom view of the handle (500), while FIGS. 5B and 5C show cross-sectional perspective views of the handle (500). As shown there, the handle (500) may comprise a snare control that includes a thumb slider (508). The thumb slider (508) may be slidable along a track (516) and may further include a pointer (510) which may be used with one or more reference markings (520) on the handle (500) to indicate a diameter of a snare loop assembly (not shown), as discussed in more detail above. The thumb slider (508) may be attached to a carriage (502), which may be operatively attached to a snare (not shown), such that movement of the thumb slider (508) along the track (516) also moves the carriage (502) and the snare.

As shown in FIGS. 5A-5C, the carriage (502) may include one or more ratchet teeth (514). As illustrated in FIG. 5B, the carriage (502) may be positioned relative to the handle (500) such that the ratchet teeth (514) engage corresponding ratchet teeth (506) of a ratchet strip (504) in the handle (500). The engagement between the ratchet teeth (514) and (506) may allow the carriage (502) to be withdrawn along the track (516), but may prevent advancement of the carriage (502) along the track. This may allow a user to freely close the snare loop assembly (e.g., by withdrawing the thumb slider (508)), but may prevent the snare loop assembly from being inadvertently opened (e.g., by a user or by expansive forces applied to the snare loop assembly by ensnared tissue). To advance the thumb slider (508) and re-open the snare loop assembly, the carriage (502) may be moved to a second position in which the ratchet teeth (514) of the carriage (502) are temporarily disengaged from the ratchet teeth (506) of the ratchet strip (504), as shown in FIG. 5C.

For example, in some variations the thumb slider (508) may comprise a button (518) which may be configured to move the carriage (502) between the engaged and disengaged positions. The button (518) may be connected to the carriage (502) such that depression of the button (518) (as illustrated in FIG. 5C) moves the carriage (502) away from the ratchet strip (504), thereby disengaging the ratchet teeth (514) of the carriage (502) from the ratchet teeth (506) of the ratchet strip (504). While the carriage (502) is disengaged, the thumb slider (508) may be advanced along the track (516) to re-open the snare loop assembly. The button (518) may be further configured such that the carriage (502) moves back into engagement with the ratchet strip (504) when the button (518) is released.

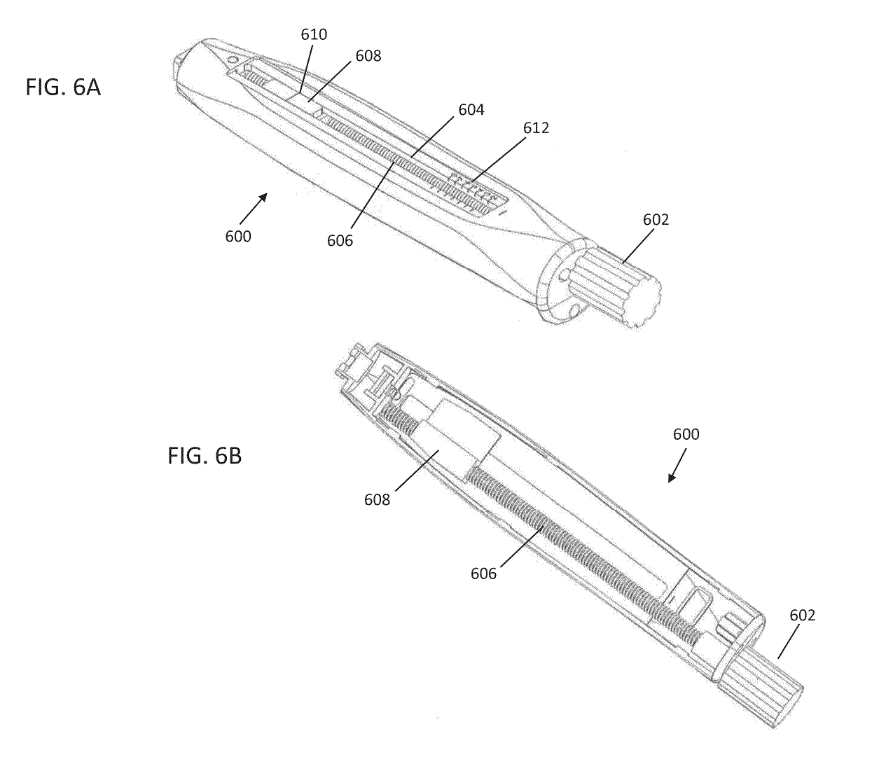

In other variations, the snare control need not comprise a thumb slider. For example, FIGS. 6A and 6B show a perspective view and a bottom cross-sectional view, respectively, of a variation of handle (600) suitable for use with the closure devices described here. As shown there, the handle (600) may comprise a control knob (602) configured to move a carriage (608) along a track (604). In the variation shown in FIGS. 6A and 6B, the control knob (602) may be connected to the carriage (608) via a lead screw (606). The control knob (602) may be rotatably connected to the lead screw (606) such that rotation of the control knob (602) also rotates the lead screw (606). The carriage (608) may be connected to the lead screw (606) such that rotation of the lead screw (606) is translated into linear movement of the carriage (608) along the track (604). The carriage (608) may in turn be operatively attached to a snare (not shown) of the snare loop assembly, such that linear movement of the carriage (608) along the track (604) also moves the snare to open and/or close the snare loop assembly. The handle (600) may further comprise one or more reference markings (612), such as those described in more detail above, and the carriage (608) may further comprise a carriage marking (610) or pointer which may align with one or more reference markings (612) to indicate the diameter of the snare loop assembly. In should be appreciated that in some variations (as will be discussed in more detail below), a handle may comprise both a thumb slider and a control knob for controlling the snare.

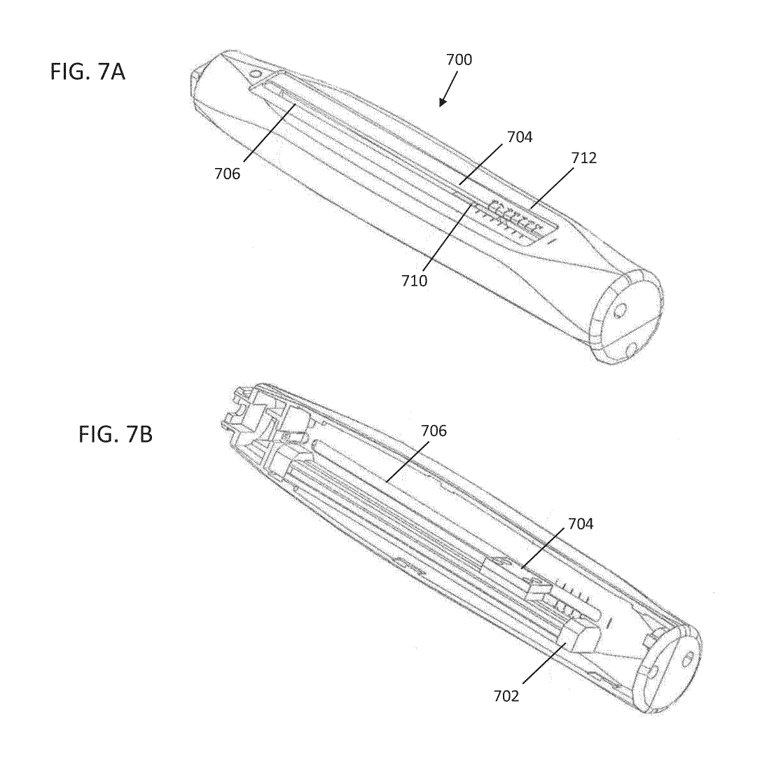

FIGS. 7A and 7B show a perspective view and a cross-sectional perspective view, respectively, of another variation of a handle (700) suitable for use with the closure devices described here. As shown there, the handle (700) may comprise a pneumatic slide (702) which may be configured to move a carriage (704) along a track (706) using pressurized air. The carriage (704) may be operatively connected to a snare (not shown), and the carriage (704) and snare may be advanced or withdrawn using the pneumatic slide (702) to open or close, respectively, the snare loop assembly. Additionally, the handle may comprise one or more reference markings (712) along the track (706) (such as one or more of the reference markings discussed in more detail above), and the carriage (704) may further comprise a carriage marking (710) or pointer which may align with one or more reference markings (712) to indicate the diameter of the snare loop assembly.

As mentioned above, when the snare loop assembly is initially closed around tissue, the diameter of the snare loop assembly (e.g., as indicated by reference markings and a snare control) may provide an indication of how much tissue has been captured by the snare loop assembly and the size of the suture loop that may be delivered from the snare loop assembly. In some variations, it may be desirable to further reduce the diameter of ensnared tissue, which may further reduce the likelihood of leak formation as well as the size of any leaks that may occur. For example, in some variations, a tensioning device (such as those described in U.S. patent application Ser. No. 13/490,919, which was previously incorporated by reference in its entirety) may be used to further tension the suture loop after the suture loop is delivered. Additionally or alternatively, it may be desirable to further tension or otherwise reduce the diameter of the snare loop assembly prior to releasing the suture loop from the snare loop assembly. In some variations, the amount of force applied during these additional tensioning steps may be dependent on the diameter of the snare loop assembly during the initial closure.

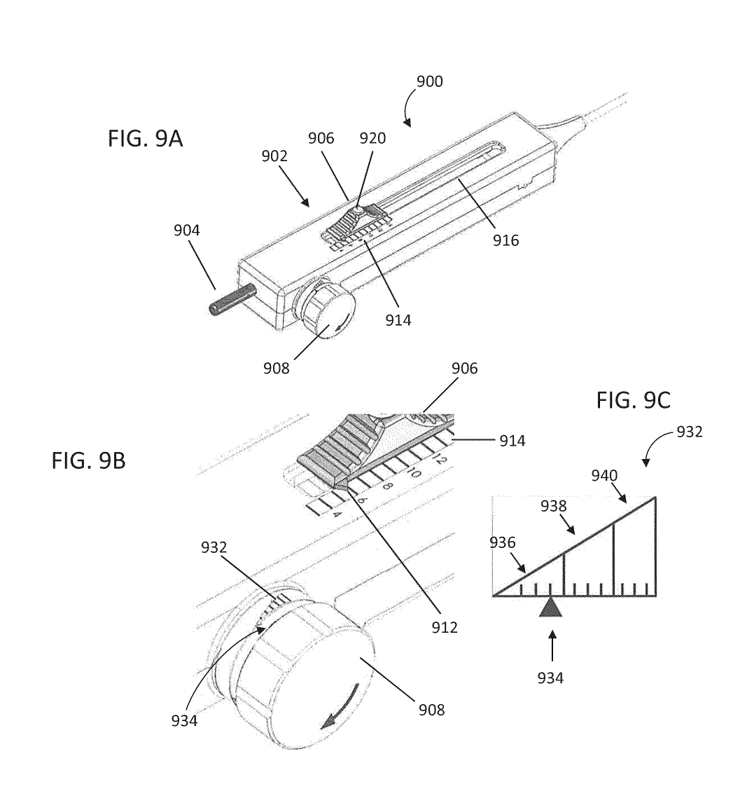

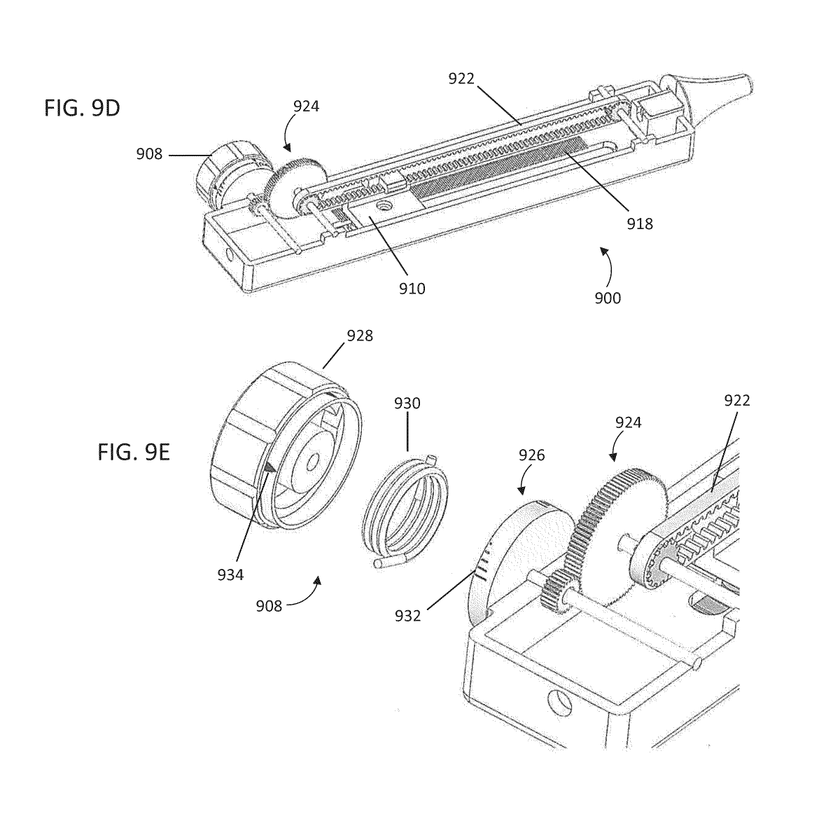

For example, FIGS. 9A-9E show one variation of a handle (900) which may be used to control the snare loop assembly as described here. As depicted in FIG. 9A, the handle (900) may comprise a snare control (902) and a suture control (904). The snare control may comprise a thumb slider (906) and a rotation knob (908). Each of the thumb slider (906) and the rotation knob (908) may be operatively attached to a carriage (910), such that either the thumb slider (906) or the rotation knob (908) may be actuated to advance or retract the carriage (910) relative to the handle (900). The carriage (910) may be operatively connected to a snare of the snare loop assembly (not shown), such that movement of the carriage (910) moves the snare relative to the elongate body to open or close the snare loop assembly, such as discussed in more detail above.

As shown in a perspective view in FIG. 9B, the thumb slider (906) may comprise a pointer (912) which may align with one or more reference markings (914) of the handle (900) to provide a measurement of the current diameter of the snare loop assembly as the thumb slider (906) is moved relative to a track (916) in the handle (900). In some variations, the carriage (910) may be configured to engage ratchet teeth of a ratchet strip (918) (as shown in a cross-sectional bottom perspective view in FIG. 9D), such as described above with respect to FIGS. 5A-5C. In these variations, the engagement between the carriage (910) and the ratchet strip (918) may allow the carriage (910) to be withdrawn along the track (916) (either by actuating the thumb slider (906) or the rotation knob (908)) to close the snare loop assembly, but may resist or otherwise prevent advancement of the carriage (910) relative to the handle. To advance the carriage (910) and open the snare loop assembly, a user may depress a button (920) in the thumb slider (906) to disengage the carriage (910) and the ratchet strip (918), and may advance the thumb slider (906) to advance the carriage (910).

As mentioned above, the suture control may include a control knob (908) configured to move the carriage (910) relative to the track (916). As shown in FIG. 9D, the carriage (910) may be connected to a toothed belt (922). The toothed belt may be connected to a gear assembly (924), which in turn may be connected to the control knob (908) such that rotation of the control knob (908) moves the toothed belt (922), and with it, the carriage (910). Accordingly, rotation of the control knob (908) may be used to open and/or close the snare loop assembly. In some variations, the control knob (908) may be configured to engage the gear assembly (924) when rotated in a first direction, but not a second direction (e.g., the control knob (908) may freewheel when rotated in the second direction). In these variations, the control knob (908) may be configured to withdraw the carriage (and close the snare loop assembly) when the control knob (908) is rotated in a first direction, but does not advance or otherwise move the carriage (910) and the snare when the control knob (908) is rotated in an opposite direction. This may allow a user to incrementally tighten the snare loop assembly with the control knob without inadvertently re-opening the snare loop assembly.

In some variations, the control knob (908) may be configured to provide a measurement or other indication of the force being applied by the control knob (908) to the snare loop assembly. For example, as shown in FIG. 9E, the control knob may comprise a base piece (926), a handle piece (928), and a spring member (930) connecting the base piece (926) and the handle piece (928). In the variation shown in FIG. 9E, the spring member (930) may comprise a torsional spring having a first leg of the torsional spring (930) connected to handle piece (928) and a second leg of the torsional spring (930) connected to the base piece (926), such that a rotational force applied to handle piece (928) may be translated to the base piece (926). This may result in rotation of the base piece (926), which may move the carriage (910) via the gear assembly (924), as discussed above. Accordingly, rotation of the control knob (908) may tighten the snare loop assembly around the captured tissue. As the snare loop assembly is tightened around tissue, rotation of the control knob (908) increases the tension on the snare. This increased tension may resist further rotation of the base piece (926). Eventually, the increased tension may overcome the spring force of the spring member (930), and the spring member (930) may begin to twist such that the first leg may rotate toward the second leg. This may cause the handle piece (928) to rotate relative to the base piece (926).

Relative rotation between the handle piece (928) and the base piece (926) may provide a measurement of the force being applied to the snare. For example, in the variation shown in FIG. 9E, the base piece (926) may comprise one or more reference markings (932) and the handle piece (928) may comprise a pointer (934) (or vice versa). The relative positioning between the pointer (934) and the reference markings (932) may be indicative of the force applied to the snare. In some variations, the reference markings (932) may provide numerical values of the tension being applied to the snare. In other variations, the reference markings (932) may be configured to correspond to one or more of the reference markings (914) associated with the track (916), as will be discussed in more detail below.

By providing a force measurement with the control knob (908), the snare loop assembly may be closed around tissue to a predetermined tension. In some instances, it may be desirable to alter this amount of tension based on the amount of tissue captured by the snare loop assembly. In some variations, the amount of force applied to the snare loop assembly may be proportional to the diameter of the snare loop assembly when initially closed. For example, it may be desirable to apply a greater force to the snare loop assembly when a larger amount of tissue has been captured by the snare loop assembly because the larger diameter of the snare loop assembly is more likely to result in the delivery of a larger suture loop (which may have a greater risk of developing leaks). Conversely, when the snare loop assembly has captured less tissue, a smaller force may be required to close the snare loop assembly to a desired diameter. Accordingly, when tightening the snare loop assembly, a user may select a tensioning force based on the diameter of the snare loop assembly.

In some variations, the diameter reference markings may comprise one or more marking regions which correspond to one or more marking regions of the control knob (908). For example, in some variations of the handle (900) shown in FIGS. 9A-9E, the reference markings (914) on the track (916) include a first region, a second region, and a third region (such as described above with respect to FIG. 4C). The first region may include a range of diameters that are larger than the diameters of the second region, which may in turn be larger than the diameters of the third region. Similarly, as illustrated in FIG. 9C, the reference markings (932) of the control knob (908) may include a first region (940), a second region (938), and a third region (936), wherein the first region (940) encompasses a range of tensioning forces that are greater than the range of tensioning forces in the second region (938), which in turn may be greater than the range of tensioning forces in the third region (936). In some embodiments, the first region (940) may encompass a range of tensioning forces between 12 and 14 lbs, the second region (938) may encompass a range of tensioning forces between 10 and 12 lbs, and the third region (936) may encompass a range of tensioning forces between 8 and 10 lbs. In another embodiment, the first region (940) may encompass a range of tensioning forces between 9 and 10 lbs, the second region (938) may encompass a range of tensioning forces between 7.5 and 9 lbs, and the third region (936) may encompass a range of tensioning forces between 6 and 7.5 lbs. The first, second, and third regions of the reference markings (914) of the track (916) may be color-coded or otherwise configured to be associated with the first region (940), the second region (938), and the third region (936), respectively. Corresponding regions may be visually linked (i.e., may have the same color, shape, pattern, etc.) so that a user may realize that the first region of the reference markings (914) on the track (916) is associated with the first region (940) of the reference markings (932) of the control knob (908), the second region of the reference markings (914) on the track (916) is associated with the second region (938) of the reference markings (932) of the control knob (908), and so on.

In use, the snare loop assembly may be initially closed down around tissue to provide an initial diameter reading of the snare loop assembly. In some variations, the initial closure may include rotating the control knob (908) or withdrawing the thumb slider (906) until the tissue captured by the snare loop assembly significantly resists further withdrawal of the snare. In other variations, the initial closure may include rotating the control knob (908) or withdrawing the thumb slider (906) until a predetermined force is applied to the snare. For example, in some variations the spring member (930) of the control knob (908) may be configured such that the handle piece (928) begins rotating relative to the base piece (926) when the predetermined force is applied to the snare. In these variations, a user may rotate the control knob (908) to tighten the snare loop assembly until the handle piece (928) begins to rotate relative to the base piece (926). In other variations, the thumb slider (906) may comprise a force gauge which may measure the force applied to the snare. In these variations, the thumb slider may be withdrawn until the force gauge indicates that the predetermined force has been reached.

Once the snare loop assembly has been initially tightened, the user may use the reference markings (914) to measure the diameter of the snare loop assembly. Depending on the measured diameter of the snare loop assembly, a user may choose to reposition the snare loop assembly, as discussed in more detail above. If the user chooses to proceed with the chosen location, the snare loop assembly may be further tightened using the control knob (908). The measured diameter of the snare loop assembly may be used to determine what force is applied to the snare during this tightening step. In some variations where the reference markings (914) of the track (916) comprise a plurality of regions that are associated with a plurality of regions of the reference markings (932) of the control knob (908), the user may tighten the snare loop assembly using a force from the force region that is associated with the region of the measured snare loop assembly diameter. For example, if the pointer (912) of the thumb slider (906) is in the first region of the reference markings (914) of the track (916), a user may rotate the control knob (908) until the pointer (934) of the control knob (908) reaches the first region (940) of the reference markings (932) of the control knob (908). If the diameter is instead in the second region of the reference markings (914) of the track (916), a user may rotate the control knob (908) until the point (934) of the control knob (908) reaches the second region (938) of the reference markings (932) of the control knob (908), and so on. Thus, the region of the reference markings (914) of the track (916) may indicate to a user which region of the reference markings (932) of the control knob (908) to use and therefore a how much force to apply. For example, the second region (938) corresponds to smaller tensioning forces than the first region (940), and therefore the tensioning force applied during tightening of the snare loop assembly may be reduced as the diameter of the snare loop assembly is reduced.

While the snare control is shown in FIGS. 9A-9C as having both a control knob and a thumb slider, it should be appreciated that in some variations the handle may comprise only a control knob or only a thumb slider. In variations where the snare control comprises only a control knob, the carriage may comprise a carriage marking or pointer which may be used with the reference markings to measure the diameter of the snare loop assembly. Conversely, in variations where the snare control comprises only a thumb slider, the thumb slider may comprise a force gauge which in some variations may include one or more reference markings that are associated with one or more reference markings of the track.

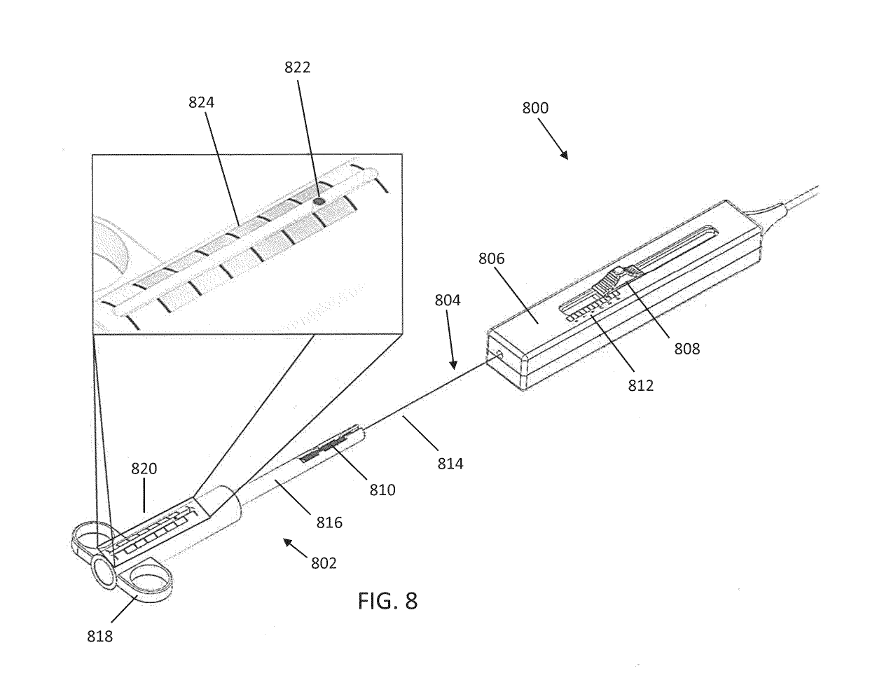

Once the snare loop assembly has been tightened, the suture loop may be tightened and released from the snare loop assembly. The suture loop may be further tightened using one or more tensioning devices, such as mentioned above. In some variations, the user may apply one or more predetermined forces to the suture loop during tightening. In some of these variations, the level of the force applied to the suture loop may be selected based on the diameter to which the snare loop assembly was closed around the tissue. For example, FIG. 8 shows a variation of a closure device (800) and a tensioning device (802) which may be used to tension a suture loop (804) of a snare loop assembly (not shown) of the closure device (800). As shown there, the closure device (800) may comprise a handle (806) having a snare control (808) and a suture control (810). The snare control (808) may be configured to advance and retract the snare of the snare loop assembly to open and close the snare loop assembly, and may include any suitable mechanism as discussed in more detail above. The handle (806) may further comprise one or more reference markings (812), which may be used in conjunction with the snare control (808) to indicate the diameter of the snare loop assembly as discussed above. The suture control (810) may be attached to a tail portion (814) of the suture loop (804), such that withdrawal of the suture control (810) tightens the suture loop (804).

The tensioning device may comprise a suture attachment mechanism (816), a handle portion (818), and a force gauge (820). The suture attachment mechanism (816) may be configured to grip, hold or otherwise attach to the suture control (810), and a user may pull the handle portion (818) away from the closure device (800) to apply a tensile force to the suture loop (804). The force gauge (820) may measure the force applied by the tensioning device (802) to the suture loop (804) (e.g., by measuring the force between the suture attachment mechanism (816) and the handle portion (818)). Generally, the force gauge (820) may comprise a pin (822) that is moveable relative to one or more reference markings (824). In some variations, one or more of the reference markings (824) of the tensioning device (802) are configured to correspond to one or more of the reference markings (812) of the handle (806). For example, when the reference markings (812) of the handle (806) comprise tick marks, the reference markings (824) of the tensioning device (802) may include tick marks that correspond to the tick marks of the handle (806). When the reference markings (812) of the handle (806) include one or more marking regions, the reference markings of the tensioning device (802) may include one or more marking regions that correspond to the marking regions of the handle (806). The reference markings may be color-coded or otherwise labeled to allow a user to determine which markings on the tensioning device (802) are associated with which markings on the handle (806). For example, in some variations where the handle (806) has reference markings that include a first region, a second region, and a third region (as discussed in more detail above), each of these regions may be labeled in a different color. In some of these variations, the reference markings of the force gauge (820) of the tensioning device (802) may also include a first region, a second region, and a third region, where the first region of the tensioning device (802) is the same color as the first region of the handle (806), the second region of the tensioning device (802) is the same color as the second region of the handle (806), and the third region of the tensioning device (802) is the same color as the third region of the handle (806).

A user may use the reference markings (812) of the handle (806) to determine what force to apply to the suture control (810) during tensioning of the suture loop (804). For example, the snare control (808) may be used to close the snare loop assembly around tissue to ligate tissue, as described in more detail above. The user may measure the diameter of the snare loop assembly using the reference markings (812), and may then tighten the suture loop (804) (e.g., using the suture control (810)) and release the suture loop (804) from the snare loop assembly. The user may then apply additional tension to the suture loop (804) using the tensioning device (802), and may apply a predetermined force to the suture loop (804). This predetermined force may be selected based on the diameter of the snare loop assembly that had been measured prior to release of the suture loop (804) from the snare loop assembly.

In some variations, the predetermined force may be proportional to the size of the diameter of the snare loop assembly (larger amounts of tissue captured by the snare loop assembly may require greater tensioning forces to reduce the suture loop to a given diameter). For example, when the reference markings (812) of the handle (806) include the first, second, and third regions discussed above with respect to FIG. 4C, the amount of tension applied to the suture loop may be dependent on whether the measured diameter is in the first, second, or third region. For example, if the measured diameter of the snare loop assembly is in the first region of the reference markings (812), a user may apply tension with the tensioning device until the pin (822) reaches a first region of the force gauge (which may correspond to a first range of forces, e.g., 12 to 14 lbs or 9 to 10 lbs)). If the measured diameter of the snare loop assembly is in the second region of the reference markings (812), a user may apply tension with the tensioning device until the pin (822) reaches a second region of the force gauge (which may correspond to a second range of forces, e.g., 10 to 12 lbs or 7.5 to 9 lbs)). If the measured diameter of the snare loop assembly is in the third region of the reference markings (812), a user may apply tension with the tensioning device until the pin (822) reaches a third region of the force gauge (which may correspond to a third range of forces, e.g., 8 to 10 lbs or 6 to 7.5 lbs)). Thus, the region of the reference markings (812) may indicate to a user which region of the reference markings (824) of the force gauge to use and therefore a how much force to apply. For example, the second region may correspond to smaller tensioning forces than the first region, and therefore the tensioning force applied during tightening of the snare loop assembly may be reduced for smaller snare loop assembly diameters.

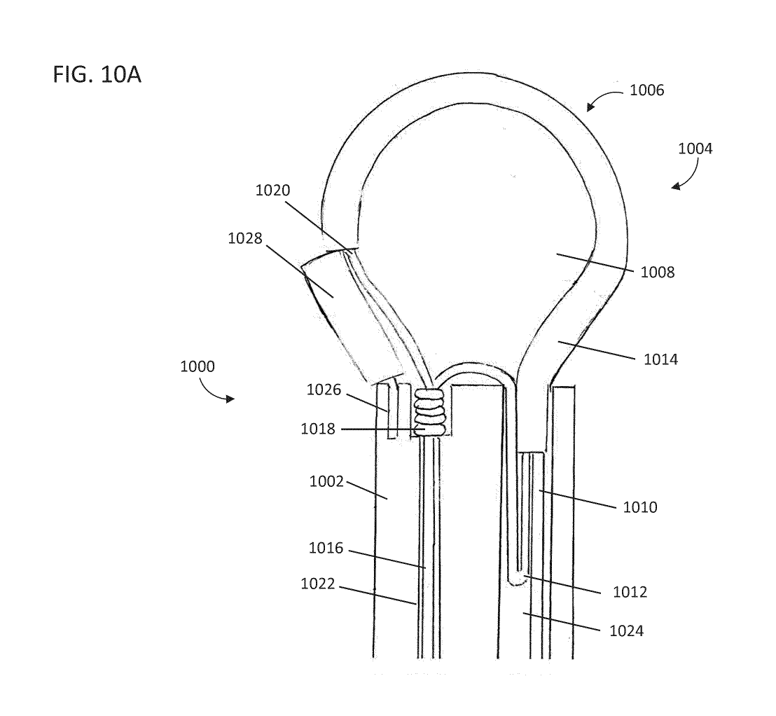

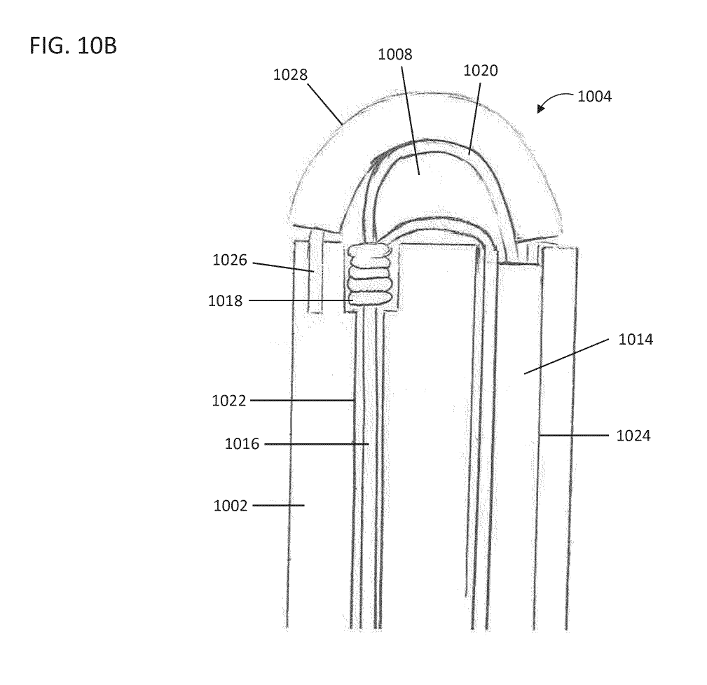

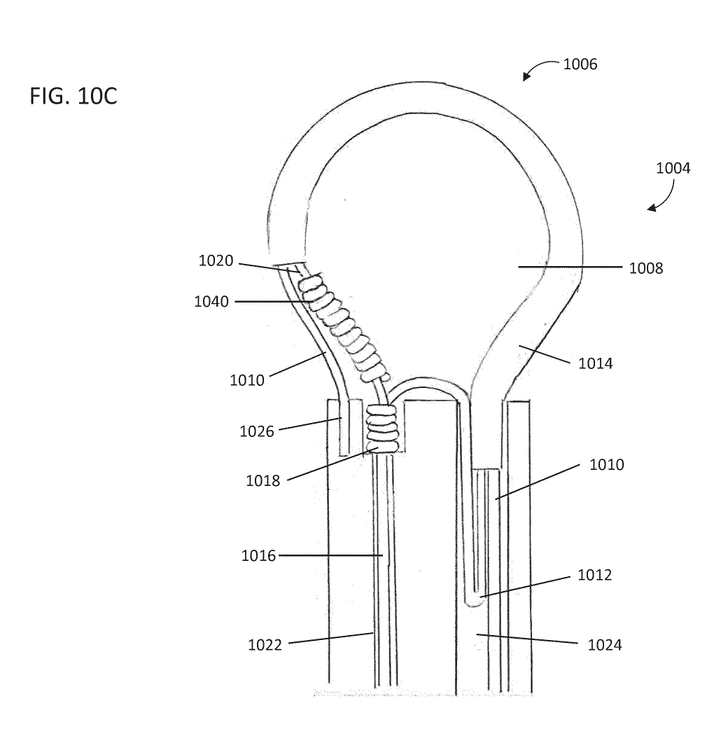

In some instances, it may be desirable to limit the amount that the snare loop assembly may be closed (i.e., set a minimum diameter for the snare loop assembly), which may reduce the risk that a snare loop assembly may be used incorrectly to cleave through tissue. In some instances, one or more portions of the handle may limit the amount the snare may be withdrawn relative to the elongate body. Additionally or alternatively, the snare loop assembly may comprise one or more structures which may limit the amount of closure of the snare loop assembly. For example, FIGS. 10A and 10B show cross-sectional side views of a distal portion of one such variation of a closure device (1000). As shown there, the closure device (1000) may comprise an elongate body (1002) and a snare loop assembly (1004), which may define a loop (1006) encircling an aperture (1008). The snare loop assembly (1004) may comprise a snare (1010), a suture loop (1012), and a retention member (1014), such as discussed in more detail above. The suture loop (1012) may include a tail portion (1016), a suture knot (1018), and a loop portion (1020). One end of the snare (1010) may extend through a first lumen (1024) in the elongate body (1002) and the tail portion (1016) of the suture loop (1012) may extend through a second lumen (1022) in the elongate body. A second end (1026) of the snare (1010) may be fixed relative to the elongate body (1002). As discussed in more detail above, movement of the snare (1010) into and out of the first lumen (1024) may increase and decrease the size of the loop (1006) defined by the snare loop assembly (1004).

Also shown in FIG. 10A is a stopper tube or coil (depicted in FIG. 10A as a tube (1028)). The stopper tube (1028) may be positioned around one or more portions of the snare loop assembly that extend from the elongate body (1002). The stopper tube (1028) may be sized and configured such that it is prevented from entering the first lumen (1024) as the snare loop assembly is closed. Accordingly, when the snare loop assembly is moved from an open configuration (such as shown in FIG. 10A) to a closed configuration (as shown in FIG. 10B), the stopper tube (1028) may be pulled toward the first lumen (1024) without actually entering the first lumen (1024). Thus, the stopper tube (1028) may prevent further withdrawal of the snare (1010), and may thereby limit the amount that the snare loop assembly (1004) may be closed.

The stopper tube (1028) may be positioned around any suitable portion of the snare loop assembly (1006). In the variation shown in FIGS. 10A and 10B, the stopper tube (1028) may be positioned around the snare (1010) between the fixed end (1026) of the snare (1010) and the retention member (1014), but not around the suture loop (1012). In these variations, the suture loop (1012) may be released form the snare loop assembly (1004) without needing to be released through the stopper tube (1028). In other variations, the stopper tube (1028) may be positioned around only a portion of the suture loop (1012), around the suture loop (1012) and the snare (1010), or around the suture loop (1012), snare (1010), and a portion of the retention member (1014). In these variations, it may be necessary to either release the stopper tube (1028) from the snare loop assembly with the suture loop (1012), or to break the engagement between the suture loop (1012) and the stopper tube (1028).

In other variations, a stopper tube or coil may be used to set a minimum diameter to which a suture loop may be tightened. For example, FIGS. 10C and 10D show another variation of closure device (1003), which may comprise a snare loop assembly (1004) and an elongate body (1002) as discussed above with respect to FIGS. 10A and 10B (identical components are labeled as such). As shown there, the snare loop assembly (1004) may comprise a stopper tube or coil (shown in FIG. 10C as coil (1040)) positioned around a segment of the loop portion (1020) of the suture loop (1012). The stopper coil (1040) may be sized and configured such that it is prevented from passing through the suture knot (1018) of the suture loop (1012). As the suture loop (1012) is tightened, the ends of the stopper coil (1040) may be pulled into contact with the suture knot (1018) (as shown in FIG. 10D), which may prevent further tightening of the suture loop (1012). In these instances, the stopper coil may also be released from the snare loop assembly with the suture loop (1012) and the length of the stopper coil (1040) may set the minimum diameter for the loop portion (1020) during tightening of the suture loop (1012). In some instances, the stopper coil (1040) may also be configured to set a minimum diameter for the snare loop assembly (e.g., the stopper coil (1040) may be sized and configured to be prevented from entering the first lumen (1024)), but need not be so configured.

In some variations, the stopper coil or tube may be formed from a radiopaque material, such that the coil or tube may be viewed using indirect visualization (such as fluoroscopy). In these variations, a user may view the stopper coil during tightening of the suture loop to estimate the diameter of the suture loop. For example, if the two ends of the stopper coil are in contact, a user may know that the diameter of the suture loop is approximately equal to the length of the stopper coil. If the two ends of the stopper coil are not in contact, a user may estimate the diameter of the suture loop based on the distance between the ends of the stopper coil.

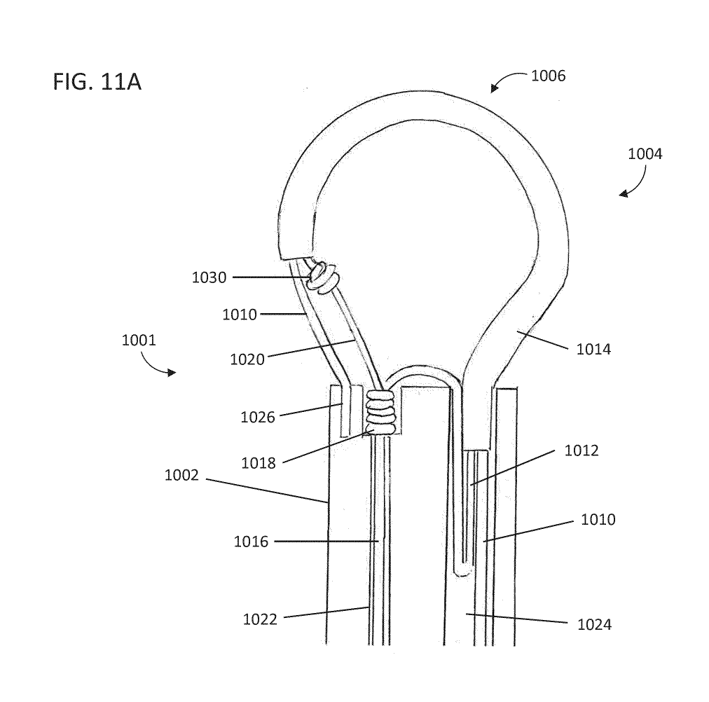

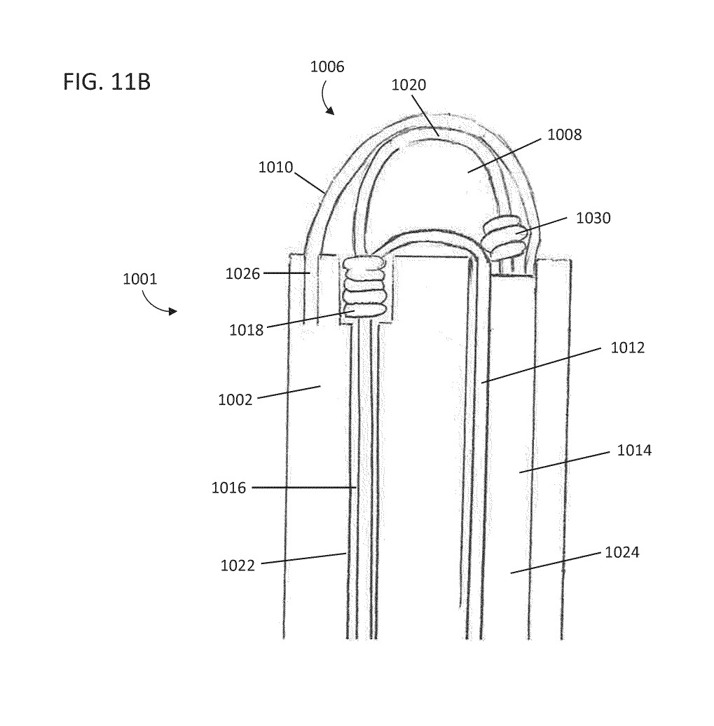

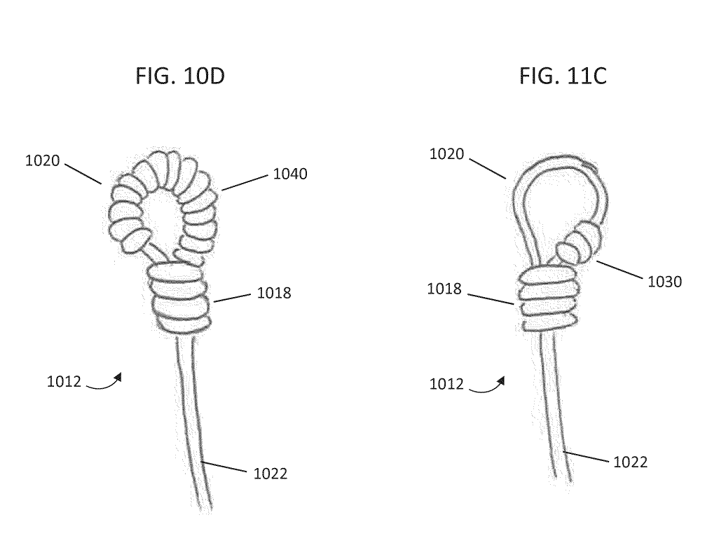

FIGS. 11A and 11B depict cross-sectional side views of another variation of a closure device (1001) as described here. The closure device (1001) may comprise a snare loop assembly (1004) and an elongate body (1002) as discussed above with respect to FIGS. 10A and 10B (identical components are labeled as such). In these variations, the snare loop assembly (1004) may comprise a knot (1030) or other stopper element (e.g., a bead, clip, or the like) attached to a portion of the snare loop assembly (1004) that extends from the elongate body (1002). For example in the variation shown in FIGS. 11A and 11B, the stopper element may be a knot (1030) formed in the loop portion (1020) of the suture loop (1012). In other variations, the stopper element may be attached to or formed in the suture loop, the snare, and/or the retention member. The stopper element (1030) may be sized and configured such that it is prevented from entering the first lumen (1024) as the snare loop assembly is moved from an open configuration (such as shown in FIG. 11A) to a closed configuration (as shown in FIG. 11B). Accordingly, when the stopper element (1030) is brought into contact with the entrance of the first lumen (1024), the stopper element (1030) may prevent further closure of the snare loop assembly, thereby setting a minimum diameter for the snare loop assembly.

When the stopper element (1030) is attached to or otherwise formed on a portion of the loop portion (1020) of the suture loop (1012), the stopper element may be configured to set a minimum diameter to which the loop portion (1020) may be tightened. For example, when the suture loop (1020) shown in FIGS. 11A and 11B is tightened, the stopper element (1030) may be pulled into contact with the suture knot (1018) of the suture loop (1012), but may be prevented from passing through the suture knot (1018) (as shown in FIG. 11C). Accordingly, the placement of the stopper element (1030) along the loop portion (1020) may set the minimum diameter to which the suture loop (1012) may be tightened. In these variations, the stopper element (1030) may also be configured to set a minimum diameter for the snare loop assembly (e.g., may be sized to be prevented from entering the first lumen (1024) of the elongate body), but need not be so configured.

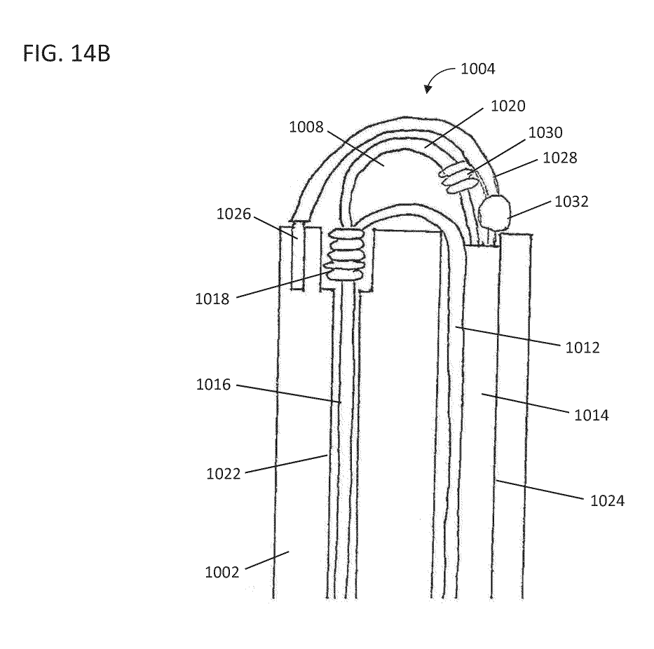

In some instances, the closure devices described here may comprise a first stopper tube, coil, or element configured to set a minimum diameter of the snare, and a second stopper tube, coil, or element configured to set a minimum diameter of the suture loop. For example, FIGS. 14A and 14B depict cross-sectional side views of such a variation of the closure device (1001). The closure device (1001) may comprise a snare loop assembly (1004) and an elongate body (1002) as discussed above with respect to FIGS. 10A and 10B and FIGS. 11A and 11B (identical components are labeled as such). In this variation, the snare loop assembly (1004) may comprise a first stopper element (1028) attached to or positioned around the snare (1010), and a second stopper element (1030) attached to or positioned around the loop portion (1020) of the suture loop (1012). In the variation shown in FIGS. 14A and 14B, the first stopper element (1028) is depicted as a tube positioned around the snare (1010) having a distal portion (1032) with an enlarged outer diameter. The second stopper element (1030) is depicted as a knot formed in the loop portion (1020) of the suture loop (1012). It should be appreciated, however, that any stopper element described above with respect to FIGS. 10A-10D and 11A-11B could be used. The first stopper element (1028) may be configured to set a minimum diameter of the snare loop assembly (1004), while the second stopper element (1030) may be configured to set a minimum diameter of the suture loop (1012). For example, the first and second stopper elements (1028, 1030) may be sized and configured such that they are prevented from entering the first lumen (1024) as the snare loop assembly is moved from an open configuration (such as shown in FIG. 14A) to a closed configuration (such as shown in FIG. 14B). Thus, the placement of the first and second stopper elements (1028, 1030) along the snare (1010) and the suture loop (1012) respectively may set a minimum diameter for each.

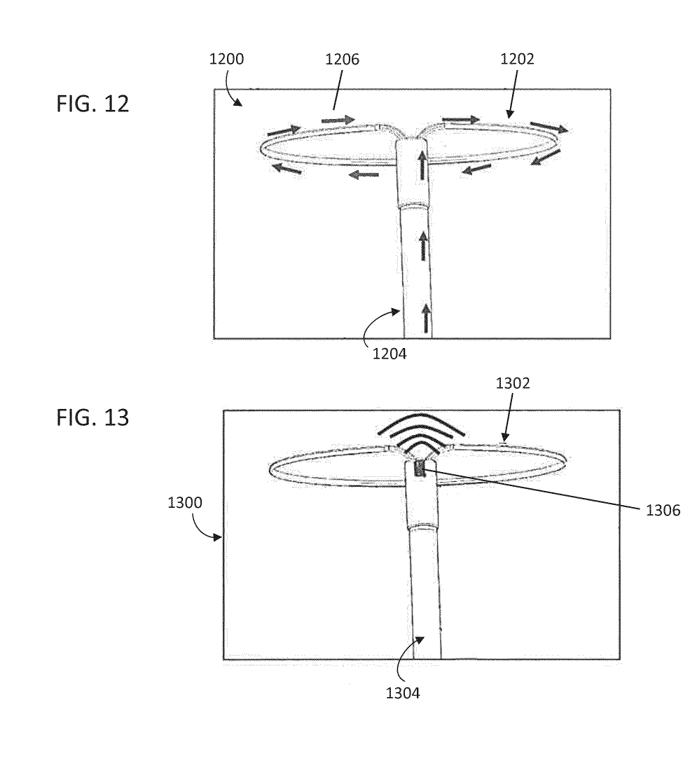

In some variations, the closure devices may be further configured to help reduce the size of the suture loop delivered by the closure device. For example, FIG. 12 shows one variation of a distal portion of a closure device (1200) as described here. As shown there, the closure device (1200) may comprise a snare loop assembly (1202) and an elongate body (1204). The snare loop assembly (1202) may be configured such that fluid (the flow of which is represented in FIG. 12 by arrows (1206)) may be advanced through the elongate body (1204) and into the snare loop assembly (1202) (e.g., through a hollow portion of the snare or another conduit extending through the snare loop assembly (1202)). In some variations, an incompressible fluid such as saline may be introduced into the snare loop assembly, which may cause radial expansion of the snare loop assembly. When the snare loop assembly has closed around tissue, this radial expansion may further close the tissue.

In other variations, one or more heated or cooled fluids may be introduced through the snare loop assembly (1202) to help reconfigure tissue. For example, fluid may be circulated through the snare loop assembly (1202), and the temperature of this fluid may be alternated between a colder temperature configured to freeze the ensnared tissue and a warmer temperature configured to thaw the ensnared tissue. The alternation between heating and thawing of the tissue may break cell walls or otherwise remove water from the cells, which may cause thinning of the ensnared tissue. As the ensnared tissue is thinned, the snare loop assembly (1202) may be closed to a smaller diameter.

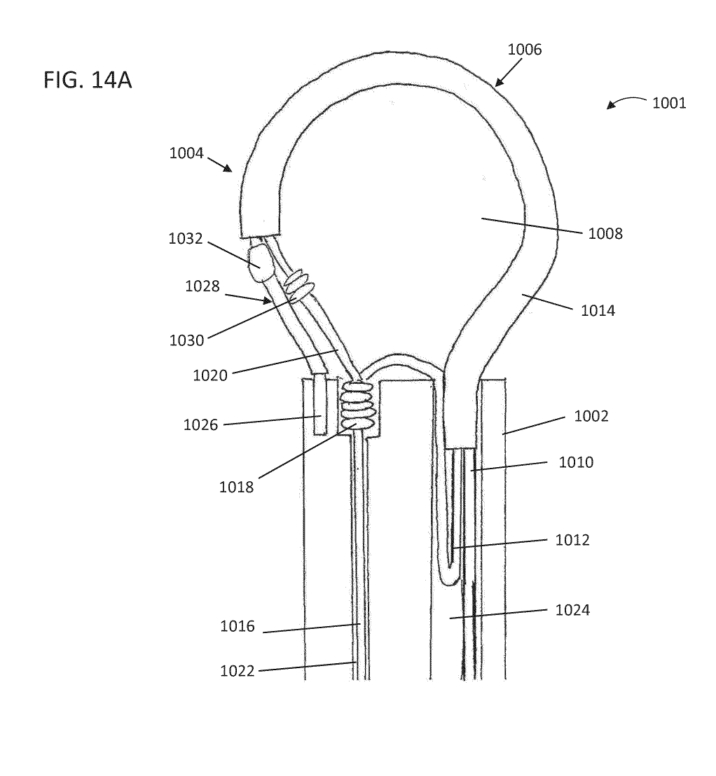

FIG. 13 shows another variation of a closure device (1300) as described here. As shown there, the closure device (1300) may comprise a snare loop assembly (1302) and an elongate body (1304). The closure device (1300) may further comprise one or more ultrasound transducers (1306) configured to apply ultrasound energy to ensnared tissue. The ultrasound energy may be configured to cause cavitation in the cells in the tissue, which may thin the tissue and allow the snare loop assembly (1302) to be further closed around the left atrial appendage. While shown in FIG. 13 as being located in the tip of elongate body (1304), the one or more ultrasound transducers (1306) may additionally or alternatively be attached to one or more outer surfaces of the elongate body and/or may be located at one or more locations around the snare loop assembly. It should be appreciated that while FIG. 13 employs ultrasound energy, any type of energy capable of causing cavitation in tissue cells may be employed (e.g., radiofrequency, microwave, laser impulses, etc.).

### II. Methods

As described here and throughout, these closure devices can be used in a number of ways (e.g., to close tissue, to measure tissue, as a diagnostic device, etc.). To close tissue, such as the left atrial appendage, with a closure device, the closure device may be advanced to the target tissue. The target tissue may be accessed in any suitable manner and any of the devices described herein may be used. In embodiments in which the target tissue is the left atrial appendage, access may be provided through a minimally invasive procedure (e.g., through a small incision above, beneath or through the rib cage, through an incision in the costal cartilage or the xiphoid, through a port, through the vasculature, etc.) or through an open surgical procedure (e.g. through a median sternotomy, mini sternotomy, thoracotomy, thoracoscopy, etc.).

Generally, methods of closing the left atrial appendage may comprise advancing a closure device to an external surface of the left atrial appendage, positioning the closure device around the left atrial appendage, closing the closure device around the left atrial appendage, delivering a suture to close the left atrial appendage and isolate it from the left atrium, and removing the closure device. The closure device may be any suitable closure device, such as a device having an elongate body with a snare loop assembly comprising a snare and a suture loop, as described above. The methods described here may further comprise determining, based on reference markings on the closure device, an appropriate diameter for the snare loop assembly to compress the tissue of the left atrial appendage and deliver the suture loop at an appropriate diameter for tissue closure. In other variations, the methods may comprise determining, based on reference markings on the closure device, an appropriate amount of force to apply to the snare loop assembly to effectuate closure of the left atrial appendage, applying the appropriate amount of force, and delivering the suture loop to close the left atrial appendage. In yet other variations, the methods may comprise determining, based on reference markings on the closure device, an appropriate amount of force to apply to the suture loop, and applying the appropriate amount of force to the suture loop to deliver it and close the left atrial appendage.

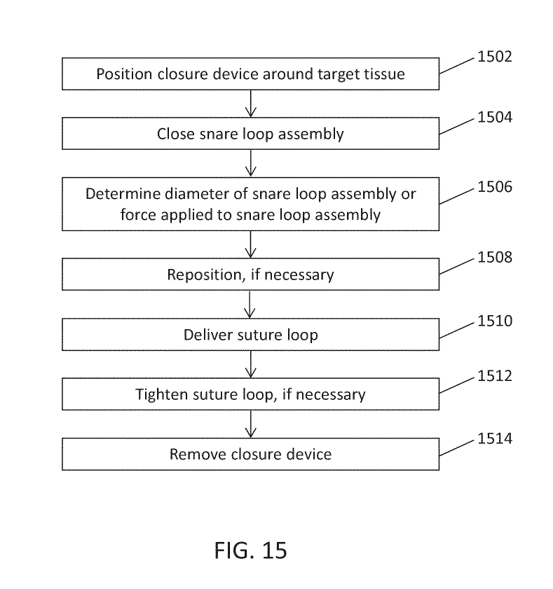

Turning now to the figures, FIG. 15 depicts a schematic illustration of a method of closing tissue using the devices of the current invention. In the embodiment depicted there, the method of closing a target tissue comprises advancing a closure device to a target tissue such that the distal end of the closure device is positioned adjacent to a surface of the target tissue. As described above, the closure device may comprise a snare loop assembly and a snare control. The snare loop assembly may comprise a snare and a suture loop. After access to the target tissue (e.g., the left atrial appendage) has been achieved, a user may position the device such that the snare loop assembly encircles the target tissue (1502). Once the user believes that the snare loop assembly is in the proper location for ligation, the user may, using the snare control, close the snare loop assembly (1504). For example, referring to FIGS. 4A and 4B, the user may close the snare loop assembly by moving the thumb slider (408) toward a proximal end of the handle (402), such that the snare is retracted relative to the elongate body (404). In some embodiments, the user may close the snare loop assembly to a predetermined diameter or may apply a predetermined force to the snare loop assembly. In other embodiments, the user may determine an appropriate diameter or applied force for the snare loop assembly during a procedure based on a tactile indication (e.g., resistance from the tissue), a visual indication (fluoroscopy or other imaging techniques), or any other suitable indication.

In variations in which a predetermined value is not used, a user may then determine the diameter of the snare loop assembly or the applied force using reference markings on the handle of the closure device (1506). Again referring to FIGS. 4A and 4B, the user may examine the reference markings (412) on the handle (402) of the closure device to determine which of the tick marks (414) the pointer (409) aligns most closely with. The tick marks (414) may be associated with a numerical scale or any other indication (e.g., other visual indication, tactile, audible, etc.) that may indicate to the user the diameter of the snare loop assembly. The user may use the diameter to confirm if the snare loop assembly is placed at the desired ligation site. In some embodiments, a user may determine that the snare loop assembly is not placed properly, and the user may reopen the snare loop assembly and reposition it (1508) (e.g., move it proximally, distally, or to another location) as many times as necessary to effectuate proper placement. In embodiments in which the user is satisfied with the location of the snare loop assembly based on its measured diameter or force, the user may deliver the suture loop (1510). In some variations, the user may tighten the suture loop (i.e. decrease its diameter) (1512) after it has been delivered, before removing the closure device (1514). It should be appreciated that using the methods described here, the user may determine the diameter of the delivered suture loop, or at a minimum, the largest possible diameter for the delivered suture loop, based on the measured diameter of the snare loop assembly.

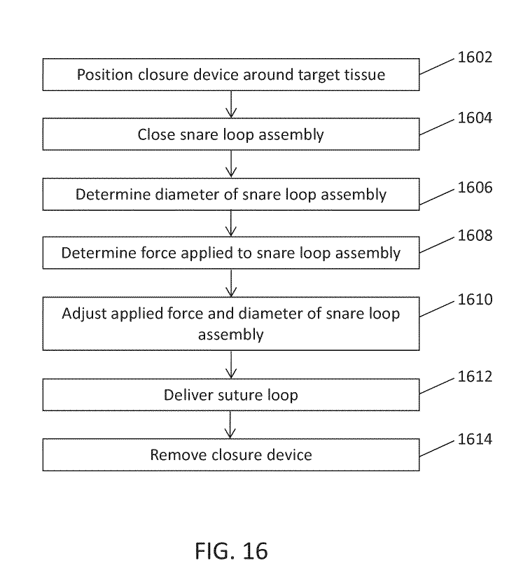

FIG. 16 schematically depicts another method of closing tissue using the closure devices described here. In this embodiment, the closure device may further comprise a control knob that may indicate to a user the force applied to the snare loop assembly based on the diameter of the snare loop assembly. As described above, the user may position the snare loop assembly around a target tissue (1602), close the snare loop assembly using the snare control (1604), and determine the diameter of the snare loop assembly using the reference markings on the closure device (1606). The user may then determine the force applied to the snare loop assembly at the chosen diameter (1608), and may, if desired, adjust the applied force and the diameter of the snare loop assembly (1610) based on the determined diameter. Once the snare loop assembly is closed to a desired diameter, or a desired force is applied, the suture loop may be delivered (1612) and the closure device may be removed (1614).

For example, as described with respect to FIGS. 9A-9E, the reference markings (914) on the handle (900) may comprise regions that correspond with, and are visually linked to, regions of the reference markings (932) of the control knob (908). The user may use the region of the reference markings (914, 932) as an indication of how much force is applied to the snare loop assembly at a specified diameter. More specifically, the user may determine, after closing the snare loop assembly initially, that the pointer (912) is in a first region of the reference markings (914) on the handle (900). The user may then look to the reference markings (932) of the control knob (908) to determine the value of the force applied to the snare loop assembly. In some embodiments, the user may adjust the applied force, and therefore the snare loop diameter, using the control knob (1610). In some variations, the thumb slider (906) and the control knob (908) may be mechanically coupled such that rotation of the control knob (908) may cause the thumb slider (906) to move, or vice versa. In these embodiments, the diameter of the snare loop assembly indicated by the reference markings (914) on the handle (900) will still accurately reflect the diameter of the snare loop assembly after rotation of the control knob (908). A user may decide how much to close the snare loop assembly based on the force applied to the snare loop assembly, the diameter of the snare loop assembly, or both. Therefore, the methods described here allow a user to achieve a snare loop assembly diameter determined to provide reliable closure while ensuring that the force applied to the snare loop assembly is maintained within a safe range to avoid tissue damage.

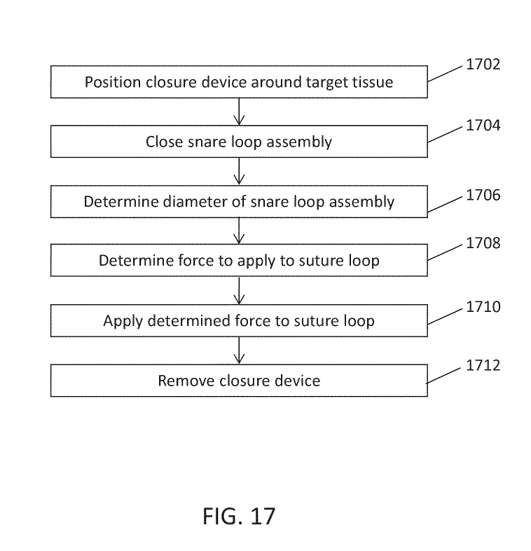

FIG. 17 schematically depicts another method of closing tissue using the closure devices described here. Similarly to the methods described with respect to FIGS. 15 and 16, the user may position the snare loop assembly around the target tissue (1702), close the snare loop assembly using the snare control (1704), and determine the diameter of the snare loop assembly using the reference markings on the closure device (1706). The user may then determine how much force to apply to the suture loop (1708) based on the diameter of the snare loop assembly and may apply the determined force to the suture loop (1710) to close the tissue. The user may then cut the tail portion (814) of the suture, if necessary, and remove the closure device (1712).

For example, referring to FIG. 8, a user may move the snare control (808) proximally until a desired snare loop assembly diameter is reached, as indicated by the reference markings (812) on handle (806). The user may then determine, based on the reference markings (812) on the handle (806) and corresponding reference markings (824) on a tensioning device, how much force should be applied to the suture loop (804) by the tensioning device to properly close the tissue. More specifically, in variations in which the reference markings (812, 824) comprise regions, a user may determine, after closing the snare loop assembly, which region is indicated by the diameter of the snare loop assembly (e.g., region 1, region 2, etc.). The user may then apply a corresponding force to the suture loop (804) through the suture control (810). In embodiments in which a tensioning device is used, the user may apply a force to the suture loop (804) until the pin (822) on the force gauge (820) of the tensioning device enters the region of the markings (824) on the force gauge (820) that corresponds with the region indicated by the diameter of the snare loop assembly. It should be appreciated that the reference markings for the suture loop need not be located on the tensioning device, and could instead be located on the tail portion of the suture. In some variations, the user may apply additional force to the suture loop (804) after it has been deployed and released from the snare loop assembly.

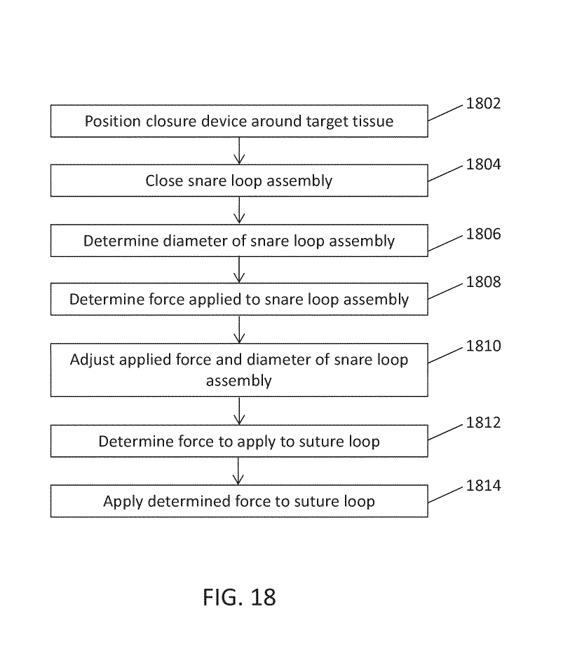

As depicted in FIG. 18, a combination of the previously described methods may also be used. As depicted there, the user may position the snare loop assembly around the target tissue (1802), close the snare loop assembly using the snare control (1804), and determine the diameter of the snare loop assembly using the reference markings on the closure device (1806). The user may then determine how much force is applied to the snare loop assembly (1808) using the reference markings on the handle of the closure device and the reference markings on the control knob. In some embodiments, the user may adjust the force applied and the diameter of the snare loop assembly (1810) based on the reference markings, as described above. Once the user is satisfied with the diameter of the snare loop assembly, and/or the force applied, the user may then determine the proper force to apply to the suture loop based on the diameter and/or applied force of the snare loop assembly (1812) and may proceed to apply the determined force to the suture loop (1814) to close the tissue.

It should also be appreciated that while the methods described above utilize the closure devices to close tissue, the devices may also be utilized as measurement or diagnostic tools. For example, the user may position the snare loop assembly around the target tissue, close the snare loop assembly using the snare control, and determine the diameter of the snare loop assembly using the reference markings on the closure device. Optionally, the user may determine the applied force using the reference markings on the closure device, as is described above. In some instances, the measured diameter and/or the measured applied force may be indicative of an ailment. For example, the measured diameter and/or the measured applied force may indicate to a user that a patient has heart disease and/or has thin or fragile tissue (for example, due to an enlarged left atrium, steroid use, etc.). In other instances, the measured diameter and/or measured applied force may be an indication that additional treatment of the tissue is necessary prior to closure. The user may use the measured diameter and/or the measured applied force to make treatment decisions for a patient. It should be appreciated that if the applied force is used, a user may wish to convert the force applied to the snare loop assembly to the force applied to the target tissue, through methods known by those of skill in the art, so that the applied force may better directly indicate the strength of the tissue.

In some embodiments, after the device is used to measure the diameter of and/or the force applied to the target tissue, the user may remove the closure device and insert a treatment device to effectuate a treatment (e.g., ablation) informed by the measured diameter and/or the measured applied force. In other embodiments, it may be unnecessary to remove the closure device before a treatment device is advanced to the target tissue. In some variations, the user may use the closure device as a treatment device (e.g., to reduce the thickness of the tissue as described with respect to FIGS. 12 and 13). The user may then effectuate the treatment. In some embodiments, the user may optionally also use the closure device to close the target tissue. For example, referring to FIGS. 12 and 13, the user may use the closure device to determine the diameter and/or applied force, treat the tissue (e.g., decrease the thickness of the tissue), and close the tissue. It should be appreciated that the user may use the closure device to close the target tissue after the user determines the diameter and/or the applied force, uses a treatment device, or both.

Optionally, the closure device may be used to measure the diameter of, and/or the force applied to, a target tissue to confirm the effectiveness of a treatment (e.g. a treatment to strengthen the tissue, reduce tissue thickness, etc.). In these embodiments, the closure device may be used to measure the diameter of, and/or the force applied to, a target tissue before and after a treatment has occurred to determine if the treatment was successful. For example, referring again to FIGS. 12 and 13, the user may use the closure device to measure the diameter of the tissue before and after heating and cooling or applying energy to the tissue, to determine if the appropriate amount of tissue thinning has occurred, and if the tissue is ready for delivery of the suture loop. Specifically, in some embodiments, the devices may be used to determine the effect of an ablation treatment on the tissue. For example, the closure device may be used to measure the diameter and/or the applied force before and after one or more ablation treatments to determine how the treatment affected the tissue, and more specifically, how it affected the diameter of the tissue and/or the applied force the tissue can withstand. Using this method, a user may be able to determine if a particular ablation technique causes inflammation instead of cavitation, or causes a lesion that thickens the tissue instead of thinning it. If a user applies different, successive ablation techniques (e.g., cryoablation, RF ablation, etc.), the user may use the closure device to determine which technique is the most effective for a desired result. In some instances, a user may use the closure device for closure, measurement, and if desired, treatment. For example, the closure device may be used to: temporarily measure the diameter of, and/or the force applied to, a target tissue, close the target tissue such that the tissue remains closed during a treatment procedure (e.g., ablation) to prevent perforation of the tissue during the treatment procedure, re-measure the tissue to determine if the tissue's properties have changed, and determine an appropriate diameter and/or force for tissue closure using the snare and/or the suture loop. For example, if the target tissue is the left atrial appendage, the closure device may be used to: measure the diameter of, and/or the applied force to, the left atrial appendage before an ablation treatment, close the left atrial appendage during ablation to prevent perforation of the left atrial appendage during the ablation treatment, measure the diameter of, and/or the force applied to, the left atrial appendage after the procedure to determine the appropriate diameter and/or force to use when delivering the snare and/or the suture for closure of the left atrial appendage, and deliver the snare/suture loop using the determined appropriate diameter and/or force to close the left atrial appendage.

We claim:

1. A system comprising:
an elongate body;
a snare loop assembly comprising a snare and a suture loop releasably attached thereto, wherein the snare loop assembly extends at least partially from the elongate body to define a loop having a diameter;
a controller, the controller comprising a snare control configured to move the snare relative to the elongate body, thereby altering the diameter of the loop, and further comprising one or more first reference markings, wherein the relative positioning between the snare control and the one or more first reference markings provides an indication of the diameter of the loop.

2. The system of claim 1, wherein the snare control comprises a thumb slider moveable along a track.

3. The system of claim 2, wherein the thumb slider comprises a point, wherein the relative positioning between the point and the one or more first reference markings provides the indication of the diameter of the loop.

4. The system of claim 1, wherein the snare control comprises a rotation knob.

5. The system of claim 4, wherein the rotation knob comprises a handle piece, a base piece, and a spring member connecting the handle piece and the base piece.

6. The system of claim 5, wherein rotation knob comprises at least one second reference marker, wherein the positioning of the at least one second reference marker is indicative of a force applied to the snare.

7. The system of claim 1 further comprising a tensioning device, wherein the tensioning device is configured to attach to a portion of the suture loop and reduce a diameter of the suture loop.

8. The system of claim 7 wherein the tensioning device comprises a force gauge comprising at least one second reference marker indicative of a force applied to the suture loop.

9. A device for closing tissue comprising:
an elongate body; and
a snare loop assembly,
wherein the snare loop assembly comprises a snare, a suture loop, and a retention member releasably coupling the snare and the suture loop,
wherein the snare loop comprises a loop portion and a suture knot, and
wherein the snare loop assembly further comprises a stopper tube or coil positioned around a segment of the loop portion between the suture knot and the retention member.

10. The device of claim 9 wherein the stopper tube or coil comprises a radiopaque material.

11. The device of claim 9 wherein the stopper tube or coil is sized and configured such that it is prevented from passing through the suture knot.

12. The device of claim 9 wherein a portion of the snare extends through a first lumen in the elongate body, and wherein the stopper tube or coil is sized and configured such that it is prevented from entering the first lumen.
